(19) United States
(12) Patent Application Publication
(10) Pub. No.: US 2025/0099515 A1
(43) Pub. Date: Mar. 27, 2025

(54) LACTOBACILLUS ACIDOPHILUS TO INCREASE AGMATINE PRODUCTION BY MICROBIOTA

(71) Applicant: SOCIETE DES PRODUITS NESTLE S.A., Vevey (CH)

(72) Inventors: MARC GARCIA-GARCERA, Ecublens (CH); CLAIRE LAURENCE LUCIE MARIE BOULANGE, Oron la Ville (CH); KARINE MEISSER REDEUIL, Pully (CH); NASHMIL EMAMI, Morges (CH)

(21) Appl. No.: 18/717,883

(22) PCT Filed: Dec. 7, 2022
(86) PCT No.: PCT/EP2022/084793

§ 371 Date: Jun. 7, 2024

Related U.S. Application Data
(60) Provisional application No. 63/287,648, filed on Dec. 9, 2021.

(51) Int. Cl. A61K 35/747; A61K 31/198

(52) U.S. Cl. CPC A61K 35/747; A61K 31/198

(57) ABSTRACT

Disclosed are probiotics, compositions and methods for producing agmatine using microbiota; and more particularly, isolated probiotics, compositions comprising the isolated probiotics, and methods for increasing production of agmatine and/or polyamines in the gastrointestinal tact of a subject, which provides to the subject certain health benefits, such as pain relief, antiaging effects, neuroprotective and antidepressant effects, improved vasodilatation and metabolic health, improved cellular health, decreased age-related memory loss, and/or longevity. The isolated probiotic is capable of colonizing and surviving in the gastrointestinal tract of the subject and capable of converting arginine to agmatine in the gastrointestinal tract of the subject.

### TECHNICAL FIELD

The present disclosure generally relates to probiotics, compositions and methods for producing agmatine using microbiota, more particularly, relates to probiotics, compositions and methods for increasing production of agmatine and/or polyamines in the gastrointestinal tract of a subject in order to provide to the subject certain health benefits such as pain relief, antiaging effects, neuroprotective and antidepressant effects, improved vasodilatation and metabolic health, improved cellular health, decreased age-related memory loss, and longevity. The composition comprises a probiotic capable of colonizing and surviving in the gastrointestinal tract of the subject and capable of converting arginine to agmatine in the gastrointestinal tract of the subject.

### BACKGROUND

Agmatine, also known as (4-aminobutyl) guanidine, is an aminoguanidine having a chemical structure as shown in Formula I below. Agmatine is a natural compound produced by decarboxylation of the amino acid, arginine, also known as decarboxylated arginine. Agmatine is one of the precursors of polyamines such as putrescine (diamine), spermidine (triamine) and spermine (tetraamine) in plant, prokaryotes and some mammalian cells.

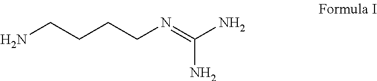

Polyamines such as putrescine, spermidine and spermine are essential for normal cell growth and viability. Spermidine is a cytoprotective and an autophagy inducer, its supplementation has been linked with antiaging effects in preclinical and clinical experiments [1, 2]. Higher systemic and urinary polyamines level are linked with growth in healthy children, but in the context of cancer, these molecules may also be associated with tumor progression [3, 4].

Besides being an intermediate of polyamines production, agmatine induces a variety of physiological and pharmacological effects on the central nervous system and other organs[5, 6]. Several health benefits have been attributed to the supplementation of synthetic agmatine. The most described and substantiated benefits are agmatine neuroprotective and antidepressant effects which are supported by several pre-clinical studies using animal model of brain ischemia, hypoxia, drug-based toxicity or behavioral test predicting antidepressant activity (tail suspension and forced swim test) [7, 8]. The effect of agmatine supplementation on pain release is also well substantiated with a clinical trial on patients suffering from radiculopathy and several pre-clinical studies [6, 7]. Early evidence (based on in-vitro or ex-vivo experiments) are found on the role of agmatine on vasodilatation, improved metabolic health (stimulation of fatty acid oxidation, decreased lipid peroxidation, improved insulin signalling) and cellular health (reduction of oxidative stress, cytoprotection, pro and antiproliferative effect) [5, 6].

Agmatine and its downstream by-products have been linked to a list of potential health benefits, including pain relief, longevity and aging improvement. To date, the supplementation of agmatine is performed through oral administration of synthetic forms of agmatine. Other alternatives to provide agmatine are through food, as derived forms of agmatine that have been found in plant-based products. However, such forms of agmatine are absorbed by the small intestine and transformed in the liver before reaching the bloodstream in the form of downstream polyamines. There is no possible direct effect of the agmatine to be evaluated as all of it is transformed. The local effects of the agmatine at the gastrointestinal level are dependent on the blood circulation, when there is the possibility to enhance microbiota-produced agmatine. Furthermore, producing synthetic agmatine is costly, compared to the cost of arginine, the upstream source molecule for agmatine. There is no proposed methodology to increase the production of agmatine by the microbiota.

Applicant of the present disclosure has identified probiotics, compositions and methods for enhancing the production of agmatine using microbiota at the gastrointestinal tract of a subject.

### SUMMARY

The present disclosure provides probiotics, compositions and methods for producing agmatine using microbiota, more particularly, relates to isolated probiotics, compositions and methods for increasing production of agmatine and/or polyamines in the gastrointestinal tract of a subject in order to provide to the subject certain health benefits such as pain relief, antiaging effects, neuroprotective and antidepressant effects, improved vasodilatation and metabolic health, improved cellular health, and longevity.

In an aspect, the present disclosure provides a method for improving pain relief, antiaging effects, neuroprotective and antidepressant effects, improved vasodilatation and metabolic health, improved cellular health, and longevity and reducing age-related memory loss of a subject in need thereof by increasing production of agmatine in a gastrointestinal tract of the subject using local microbiota, the method comprising administer to the subject a composition, the composition comprising: an isolated probiotic; and arginine, wherein the isolated probiotic is a bacterial strain having at least 90%, preferably at least 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the composition further comprises one or more of a starch source, a protein source, lipid source, a prebiotic source (such as FOS, GOS), vitamins, sugars, salt, spices, seasonings, minerals, and flavoring agents.

In an embodiment, the isolated probiotic is a Lactobacillus acidophilus strain.

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic catalyzes the production of agmatine from arginine in the gastrointestinal tract of the subject using arginine decarboxylase (ADC) and local microbiota, wherein a concentration of the agmatine in the gastrointestinal tract of the subject is at least 20 μM at 24 hours after administering the composition.

In an embodiment, the concentration of the agmatine in the gastrointestinal tract of the subject is at least 100 μM at about 24 hours after administering the composition.

In an embodiment, the composition is in a form of a dried powder, and the isolated probiotic is filled into the composition.

In an embodiment, the isolated probiotic is active in the composition.

In an embodiment, the composition is in a form of a capsule.

In an embodiment, the isolated probiotic in the capsule has a capability of surviving in a pH 1.5 fluid environment for at least 30 minutes.

In an embodiment, the isolated probiotic in the capsule is capable of surviving in a pH 3.5 fluid environment for at least 90 minutes.

In an embodiment, the isolated probiotic is capable of surviving in a pH 1.5 fluid environment for at least 10 minutes.

In an embodiment, the isolated probiotic is capable of surviving in a pH 3.5 fluid environment for at least 60 minutes.

In an embodiment, the gastrointestinal tract of the subject is a lower gastrointestinal tract of the subject.

In an embodiment, the gastrointestinal tract of the subject is large intestine of the subject.

In an embodiment, the composition is administered to the subject in an effective amount to provide the subject a daily dose of the isolated probiotic in an amount of at least 7 million CFU and a daily dose of the arginine in an amount of at least 3 g/L.

In an embodiment, wherein the subject is a human.

In an embodiment, the method further comprises administering to the subject a prebiotic before or after administering the composition.

In an aspect, the present disclosure provides a composition for improving pain relief, antiaging effects, neuroprotective and antidepressant effects, improved vasodilatation and metabolic health, improved cellular health and longevity and reducing age-related memory loss of a subject in need thereof by increasing production of agmatine in a gastrointestinal tract of the subject using local microbiota, the composition comprising: an isolated probiotic; and arginine, wherein the isolated probiotic is a bacterial strain having at least 90%, preferably at least 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396), wherein the isolated probiotic has a capability of colonizing and surviving in the gastrointestinal tract of the subject and producing agmatine from at least 20 μM arginine in the gastrointestinal tract of the subject using local microbiota.

In an embodiment, the composition further comprises one or more of a starch source, a protein source, lipid source, prebiotic source, vitamins, sugars, salt, spices, seasonings, minerals, and flavoring agents.

In an embodiment, the isolated probiotic is active in the composition.

In an embodiment, the isolated probiotic is capable of surviving in a pH 1.5 fluid environment for at least 10 minutes.

In an embodiment, the isolated probiotic is capable of surviving in a pH 3.5 fluid environment for at least 60 minutes.

In an embodiment, the composition is in a form of a capsule.

In an embodiment, the isolated probiotic in the capsule is capable of surviving in a pH 1.5 fluid environment for at least 30 minutes.

In an embodiment, the isolated probiotic in the capsule is capable of surviving in a pH 3.5 fluid environment for at least 90 minutes.

In an embodiment, the composition is in a form of a dried powder, and the isolated probiotic is filled into the composition.

In an embodiment, the isolated probiotic is a Lactobacillus acidophilus strain.

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an aspect, the present disclosure provides an isolated probiotic for improving pain relief, antiaging effects, neuroprotective and antidepressant effects, improved vasodilatation and metabolic health, improved cellular health and longevity and reducing age-related memory loss of a subject in need thereof by increasing production of agmatine in a gastrointestinal tract of the subject using local microbiota. The isolated probiotic is a bacterial strain having at least 90%, preferably 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396), wherein the isolated probiotic has a capability of colonizing and surviving in the gastrointestinal tract of the subject, and producing at least μM agmatine from arginine in the gastrointestinal tract of the subject using local microbiota.

In an embodiment, the isolated probiotic is a bacterial strain selected from a group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic is capable of producing agmatine from arginine in the gastrointestinal tract of the subject using arginine decarboxylase (ADC) and local microbiota at a pH between about 4.0 and about 8.0.

In an embodiment, the isolated probiotic has a capability of increasing the production of agmatine from arginine in the gastrointestinal tract of the subject using local microbiota by at least 50%.

In an embodiment, the isolated probiotic has a capability of increasing the production of agmatine from arginine in the gastrointestinal tract of the subject using arginine decarboxylase (ADC) and local microbiota in presence of co-factor pyridoxal-5′-phosphate (PLP) by at least 50%.

In an embodiment, the isolated probiotic has the capability of increasing the bioavailability of Agmatine, produced from arginine in the gastrointestinal tract of the subject, through delaying the transformation of Agmatine to downstream polyamines, by at least 24 hours.

In an embodiment, the isolated probiotic has a capability of producing at least 20 μM agmatine from arginine in the gastrointestinal tract of the subject using arginine decarboxylase (ADC) and local microbiota after 24 hours in the gastrointestinal tract of the subject.

In an embodiment, the isolated probiotic has a capability of surviving in a pH 1.5 environment for at least 10 minutes.

In an embodiment, the isolated probiotic has a capability of surviving in a pH 3.5 environment for at least 60 minutes.

In an embodiment, the gastrointestinal tract of the subject is a lower gastrointestinal tract of the subject.

In an embodiment, the gastrointestinal tract of the subject is large intestine of the subject.

In an embodiment, the isolated probiotic is active.

In an aspect, the present disclosure provides an isolated probiotic for improving pain relief, antiaging effects, neuroprotective and antidepressant effects, improved vasodilatation and metabolic health, improved cellular health and longevity and reducing age-related memory loss of a subject in need thereof by increasing production of agmatine in a gastrointestinal tract of the subject using local microbiota. The isolated probiotic has a capability of colonizing and surviving in the gastrointestinal tract of the subject, and producing at least 20 μM agmatine from arginine in the gastrointestinal tract of the subject having a pH ranging from about 4 to about 8 using arginine decarboxylase (ADC) and local microbiota after 24 hours in the gastrointestinal tract of the subject in the presence of arginine.

In an aspect, the present disclosure provides a method for improving pain relief, antiaging effects, neuroprotective and antidepressant effects, improved vasodilatation and metabolic health, improved cellular health, and longevity and reducing age-related memory loss of a subject in need thereof by increasing production of agmatine in a gastrointestinal tract of the subject using local microbiota. The method comprises administer to the subject a composition, the composition comprising: an isolated probiotic; and arginine, wherein the isolated probiotic has a capability of colonizing and surviving in the gastrointestinal tract of the subject, and producing at least 20 μM agmatine from arginine in the gastrointestinal tract of the subject having a pH ranging from about 5 to about 8 using arginine decarboxylase (ADC) and local microbiota at 24 hours after administering the composition to the subject.

In an aspect, the present disclosure provides a composition for improving pain relief, antiaging effects, neuroprotective and antidepressant effects, improved vasodilatation and metabolic health, improved cellular health and longevity and reducing age-related memory loss of a subject in need thereof by increasing production of agmatine in a gastrointestinal tract of the subject using local microbiota. The composition comprises: an isolated probiotic; and arginine, wherein the isolated probiotic has a capability of colonizing and surviving in the gastrointestinal tract of the subject and producing at least 20 μM agmatine from arginine in the gastrointestinal tract of the subject having a pH ranging from about 5 to about 8 using arginine decarboxylase (ADC) and local microbiota during 24 hours after administering the composition to the subject.

In an embodiment, the subject may be a mammal, preferably a human including adults and children. In an embodiment, the isolated probiotic is capable of colonizing and surviving in the gastrointestinal tract of the subject and capable of converting arginine to agmatine in the gastrointestinal tract of the subject. In an embodiment, the composition comprises a probiotic and arginine.

In an aspect, the present disclosure provides an isolated probiotic capable of colonizing and surviving in a gastrointestinal tract of a subject, the isolated probiotic is capable of producing agmatine in the gastrointestinal tract of the subject using microbiota. In an embodiment, the isolated probiotic is capable of producing agmatine from arginine in the gastrointestinal tract or a lower gastrointestinal tract of the subject.

In an embodiment, the isolated probiotic is a bacterial strain capable of colonizing and surviving in the gastrointestinal tract of the subject, and capable of converting arginine to agmatine and/or polyamines such as putrescine, spermidine and spermine at the gastrointestinal tract of the subject. In an embodiment, the isolated probiotic is a bacterial strain capable of converting arginine to agmatine and/or polyamines such as putrescine, spermidine and spermine using arginine decarboxylase (ADC). In an embodiment, the isolated probiotic is a bacterial strain capable of converting arginine to agmatine and/or polyamines such as putrescine, spermidine and spermine using ornithine decarboxylase (ODC).

In an embodiment, the isolated probiotic is a bacterial strain capable of colonizing and surviving in the gastrointestinal tract of the subject. In an embodiment, the isolated probiotic is capable of converting arginine to agmatine in the gastrointestinal tract of the subject. In an embodiment, the isolated probiotic is a bacterial strain capable of converting arginine to agmatine using arginine decarboxylase (ADC). In an embodiment, the isolated probiotic is a bacterial strain capable of boosting the production of arginine decarboxylase (ADC) and converting arginine to agmatine using ADC in the gastrointestinal tract of the subject.

In an embodiment, the isolated probiotic is Lactobacillus acidophilus. In an embodiment, the Lactobacillus acidophilus is capable of converting arginine to agmatine using arginine decarboxylase (ADC). In an embodiment, the Lactobacillus acidophilus is a strain comprises the endogenous enzyme arginine decarboxylase (ADC). In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396).

In an embodiment, the isolated probiotic is a bacterial strain having at least 90%, 91%, 92%, 93%, 94%, 95%, 96%, 97%, 98%, 99%, 99.5% or 99.9% sequence identity to one of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396).

In an embodiment, the isolated probiotic is a Lactobacillus acidophilus strain has at least 90%, 91%, 92%, 93%, 94%, 95%, 96%, 97%, 98%, 99%, 99.5% sequence identity to one of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396).

In an embodiment, the isolated probiotic is capable of converting arginine to agmatine in the gastrointestinal tract of the subject at the pH of at least about 4.0, at least about 5.0, at least about 6.0, between about 5.0 and about 9.0, between about 6.0 and about 8.0, or about 7.0.

In an embodiment, the isolated probiotic is capable of converting arginine to agmatine in the gastrointestinal tract of the subject.

In an embodiment, the isolated probiotic is capable of converting arginine to agmatine in the gastrointestinal tract of the subject to achieve an agmatine concentration of at least 5 μM, at least 10 μM, at least 20 μM, at least 30 μM, at least 40 μM, at least 50 μM, at least 60 μM, at least 70 μM, at least 80 μM, at least 90 μM, at least 95 μM, at least 100 μM, at least 105 μM, at least 110 μM, at least 115 μM, at least 120 μM, at least 125 μM, at least 130 μM, at least 200 μM; at least 300 μM, at least 400 μM, at least 500 μM; at least 600 μM, at least 700 μM, or at least 800 μM at 24 hours after administering the composition.

In an embodiment, the isolated probiotic is capable of surviving the gastric acid environment of the subject for at least 5 minutes, at least 10 minutes, at least 20 minutes, at least 30 minutes, at least 40 minutes, at least 50 minutes, at least 60 minutes, or at least 70 minutes.

In an embodiment, the isolated probiotic is capable of surviving in a pH 2.6 environment for at least 10 minutes, at least 20 minutes, at least 30 minutes, at least 40 minutes, at least 50 minutes, at least 60 minutes, or at least 70 minutes.

In an embodiment, the isolated probiotic is capable of surviving in a pH 3.4 environment for at least 60 minutes.

In an embodiment, the subject is a human. The gastric acid in the human stomach has a pH of about 1.5 to 3.5. The isolated probiotic is capable of surviving the gastric acid environment of the human stomach.

In an aspect, the present disclosure provides a composition for increasing the production of agmatine and/or polyamines in a body part of a subject using microbiota in order to provide to the subject certain health benefits such as pain relief, antiaging effects, neuroprotective and antidepressant effects, improved vasodilatation and metabolic health, improved cellular health, and longevity.

In an embodiment, the present disclosure provides a composition for improving pain relief, antiaging effects, neuroprotective and antidepressant effects, vasodilatation and metabolic health, cellular health, and longevity in a subject in need thereof. The composition comprises a microorganism or bacteria. In an embodiment, the microorganism is a probiotic. In an embodiment, the isolated probiotic is capable of increasing the production of agmatine and/or polyamines in a body part of the subject. In an embodiment, the isolated probiotic is capable of colonizing and surviving in the gastrointestinal tract of the subject.

In an embodiment, the body part of the subject is the gastrointestinal tract, a lower gastrointestinal tract, an intestine, a small intestine or a large intestine of the subject. In an embodiment, the production of agmatine and/or polyamines is at the gastrointestinal tract of the subject. In an embodiment, the production of agmatine is at the lower gastrointestinal tract of the subject. In an embodiment, the production of agmatine is at the small intestine of the subject.

In an embodiment, the production of agmatine is at the large intestine of the subject. In an embodiment, the isolated probiotic is capable of colonizing and surviving in the gastrointestinal tract of the subject, and further capable of increasing the production of agmatine in gastrointestinal tract of the subject.

In an embodiment, the composition further comprises arginine. In an embodiment, the isolated probiotic is a bacterial strain capable of converting arginine to agmatine and/or polyamines such as putrescine, spermidine and spermine at the gastrointestinal tract of the subject. In an embodiment, the isolated probiotic is a bacterial strain capable of converting arginine to agmatine and/or polyamines such as putrescine, spermidine and spermine using arginine decarboxylase (ADC). In an embodiment, the isolated probiotic is a bacterial strain capable of converting arginine to agmatine and/or polyamines such as putrescine, spermidine and spermine using ornithine decarboxylase (ODC). In an embodiment, the isolated probiotic is a bacterial strain capable of boosting the production of arginine decarboxylase (ADC) and converting arginine to agmatine using ADC in the gastrointestinal tract of the subject.

In an embodiment, the isolated probiotic is Lactobacillus acidophilus. In an embodiment, the composition comprises Lactobacillus acidophilus and arginine. In an embodiment, the Lactobacillus acidophilus is capable of converting arginine to agmatine and/or other polyamines such as putrescine, spermidine and spermine using arginine decarboxylase (ADC). In an embodiment, the Lactobacillus acidophilus is a strain comprises the endogenous enzyme arginine decarboxylase (ADC).

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic is a bacterial strain having at least 90%, 91%, 92%, 93%, 94%, 95%, 96%, 97%, 98%, 99%, 99.5% or 99.9% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain has at least 90%, 91%, 92%, 93%, 94%, 95%, 96%, 97%, 98%, 99%, 99.5% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

The concentration of agmatine produced from arginine in the gastrointestinal tract of the subject depends on the subject. In an embodiment, the concentration of the agmatine in the gastrointestinal tract of the subject is at least 5 μM, at least 10 μM, at least 20 μM, at least 30 μM, at least 40 μM, at least 50 μM, at least 60 μM, at least 70 μM, at least 80 μM, at least 90 μM, at least 95 μM, at least 100 μM, at least 105 μM, at least 110 μM, at least 115 μM, at least 120 μM, at least 125 μM, at least 130 μM, at least 200 μM; at least 300 μM, at least 400 μM, at least 500 μM; at least 600 μM, at least 700 μM, or at least 800 μM at 24 hours after administering the composition.

In an embodiment, the composition further comprises one or more of a starch source, a protein source and lipid source.

Suitable starch sources are, for example, grains and legumes such as corn, rice, wheat, barley, oats, soy, and mixtures of these.

Suitable protein sources may be selected from any suitable animal or vegetable protein source, for example meat and meal, poultry meat or meal, fish meat or meal, soy protein concentrates, milk proteins, gluten, and the like.

Suitable lipid sources include meats, animal fats and vegetable oils or fats.

The choice of the starch, protein and lipid sources will be largely determined by the nutritional needs of the subject, palatability considerations, and the type of product applied.

Further, various other ingredients, for example, sugar, salt, spices, seasonings, vitamins, minerals, flavoring agents, fats and the like may also be incorporated into the composition as desired.

In an embodiment, the composition is in a form of a dried powder, a capsule, a shelf stable liquid, or a wet, chilled or shelf stable paste. In an embodiment, the composition is a powder. In an embodiment, the composition is in a form of a capsule. In an embodiment, the composition is a dried powder and the isolated probiotic bacteria is coated onto or filled into the composition.

In an embodiment, the isolated probiotic bacterial is active in the final composition.

In an embodiment, the isolated probiotic is capable of surviving the gastric acid environment of the subject for at least 10 minutes, at least 20 minutes, at least 30 minutes, at least 40 minutes, at least 50 minutes, at least 60 minutes, or at least 70 minutes.

In an embodiment, the isolated probiotic is capable of surviving in a pH 2.6 environment for at least 10 minutes.

In an embodiment, the isolated probiotic is capable of surviving in a pH 3.4 environment for at least 60 minutes.

In an embodiment, the subject is a human. The gastric acid in the human stomach has a pH of about 1.5 to 3.5. The isolated probiotic is capable of surviving the gastric acid environment of the human stomach for at least 10 minutes, at least 20 minutes, at least 30 minutes, at least 40 minutes, at least 50 minutes, at least 60 minutes, or at least 70 minutes.

In an embodiment, the composition is in a form of a capsule. In an embodiment, the isolated probiotic is capable of surviving the gastric acid environment of the human for at least 10 minutes, at least 20 minutes, at least 30 minutes, at least 40 minutes, at least 50 minutes, at least 60 minutes, or at least 70 minutes.

In an embodiment, the capsule enables the isolated probiotic to survive the gastric acid environment of the human for at least 0 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 2 hours, at least 3 hours, or at least 4 hours.

In an aspect, the present disclosure provides a method for increasing the production of agmatine and/or polyamines in a body part of a subject using microbiota in order to provide to the subject certain health benefits such as pain relief, antiaging effects, neuroprotective and antidepressant effects, improved vasodilatation and metabolic health, improved cellular health, and longevity. The method comprising administering to the subject a composition.

In an aspect, the present disclosure provides a method improving pain relief, antiaging effects, neuroprotective and antidepressant effects, improved vasodilatation and metabolic health, improved cellular health, and longevity in need thereof by increasing the production of agmatine and/or polyamines in a body part of a subject using microbiota, the method comprising administer to the subject a composition discussed herein above and elsewhere in the present disclosure.

In an embodiment, the method further comprises administering the composition in an effective amount to the subject to provide at least 500-700 mg arginine per day and/or at least 5-7 million CFU of the isolated probiotic per day for at least 1 month, at least 3 months, or at least 6 months.

In an embodiment, the method further comprises administering the composition in an effective amount to provide the subject a daily dose of the isolated probiotic in an amount of at least 7 million CFU and a daily dose of the arginine in an amount of at least 3 g/L of the composition.

In an embodiment, the composition comprises a probiotic capable of colonizing and surviving in the gastrointestinal tract of the subject, and further capable of increasing the production of agmatine in gastrointestinal tract of the subject.

In an embodiment, the composition further comprises arginine.

In an embodiment, the body part of the subject is the gastrointestinal tract of the subject. In an embodiment, the production of agmatine and/or polyamines is at the gastrointestinal tract of the subject. In an embodiment, the production of agmatine is at the lower gastrointestinal tract of the subject. In an embodiment, the production of agmatine is at the lower gastrointestinal tract of the subject. In an embodiment, the production of agmatine is at the small intestine of the subject. In an embodiment, the production of agmatine is at the large intestine of the subject.

In an embodiment, the isolated probiotic is a bacterial strain capable of converting arginine to agmatine and/or polyamines such as putrescine, spermidine and spermine at the gastrointestinal tract of the subject. In an embodiment, the isolated probiotic is a bacterial strain capable of converting arginine to agmatine and/or polyamines such as putrescine, spermidine and spermine using arginine decarboxylase (ADC). In an embodiment, the isolated probiotic is a bacterial strain capable of converting arginine to agmatine and/or polyamines such as putrescine, spermidine and spermine using omithine decarboxylase (ODC). In an embodiment, the isolated probiotic is a bacterial strain capable of boosting the production of arginine decarboxylase (ADC) and converting arginine to agmatine using ADC in the gastrointestinal tract of the subject.

In an embodiment, the isolated probiotic is Lactobacillus acidophilus. In an embodiment, the composition comprises Lactobacillus acidophilus and arginine. In an embodiment, the Lactobacillus acidophilus is capable of converting arginine to agmatine and/or other polyamines such as putrescine, spermidine and spermine using arginine decarboxylase (ADC). In an embodiment, the Lactobacillus acidophilus is a strain comprises the endogenous enzyme arginine decarboxylase (ADC).

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic bacteria is a Lactobacillus acidophilus.

In an embodiment, the isolated probiotic is a bacterial strain having at least 90%, 91%, 92%, 93%, 94%, 95%, 96%, 97%, 98%, 99%, 99.5% or 99.9% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain has at least 90%, 91%, 92%, 93%, 94%, 95%, 96%, 97%, 98%, 99%, 99.5% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the composition is in a form of a dried powder, a capsule, a shelf stable liquid, or a wet, chilled or shelf stable paste. In an embodiment, the composition is a powder. In an embodiment, the composition is in a form of a capsule. In an embodiment, the composition is a dried powder and the isolated probiotic is coated onto or filled into the composition. In an embodiment, the isolated probiotic bacterial is active in the composition.

In an embodiment, the concentration of the agmatine in the gastrointestinal tract of the subject is at least 5 μM, at least 10 μM, at least 20 μM, at least 30 μM, at least 40 μM, at least 50 μM at least 60 μM at least 70 μM at least 80 μM at least 90 μM at least 95 μM at least 100 μM, at least 105 μM, at least 110 μM, at least 115 μM, at least 120 μM, at least 125 μM, or at least 130 μM at about 24 hours after administering the composition.

In an embodiment, the isolated probiotic is capable of surviving the gastric acid environment of the subject for at least 10 minutes, at least 20 minutes, at least 30 minutes, at least 40 minutes, at least 50 minutes, at least 60 minutes, or at least 70 minutes.

In an embodiment, the isolated probiotic is capable of surviving in a pH 2.6 environment for at least 10 minutes.

In an embodiment, the isolated probiotic is capable of surviving in a pH 3.4 environment for at least 60 minutes.

In an embodiment, the subject may be a mammal, preferably a human including adults and children.

### BRIEF DESCRIPTION OF THE DRAWINGS

FIG. 1 illustrates the substrate and enzymes involved in agmatine homeostasis[34].

FIG. 2 illustrates the contents of the polyamines agmatine (AGM), putrescine (PUT), cadaverine (CAD), spermidine (SPD), spermine (SPM) in seeds, sprouts and microgreens of alfalfa[36].

FIG. 3 illustrates the polyamine metabolism and transport in mammalian cells and microbiota.

FIG. 4 illustrates the hybrid mechanism for putrescine production pathway consisting of a cooperation between bacteria with acid-resistance system and bacteria with ATP synthesis system [19].

FIG. 5 illustrates the test results on the effects of agmatine production using a combination of arginine and FOS of the experimental study in Example 1 disclosed herein.

FIG. 6 illustrates the test results on evolution of agmatine concentration over time (T5 h, T24 h, T48 h) with strain screening of the experimental study in Example 2 disclosed herein.

FIG. 7 illustrates the test results on tube and batch fermentation effects on the agmatine concentration of the experimental study in Example 3 disclosed herein.

FIGS. 8A and 8B illustrate the test results on effects of strain with various donors on agmatine production: A represents the overall scale, and B represent a zoom scale from 0 to 50 μM of the experimental study in Example 4 disclosed herein.

FIG. 9 illustrates the test results on the impact of different pH conditions on agmatine concentration of the experimental study in Example 5 disclosed herein.

FIGS. 10 and 11 illustrate the test results on the effects of various specific strains incubation with donor 1 on agmatine concentration of the experimental study in Example 6 disclosed herein.

FIG. 12 illustrates the test results of the effects of four different Lactobacillus acidophilus strains on the concentrations and bioavailability of agmatine using in vitro fermentation of arginine of the experimental study in Example 7 disclosed herein.

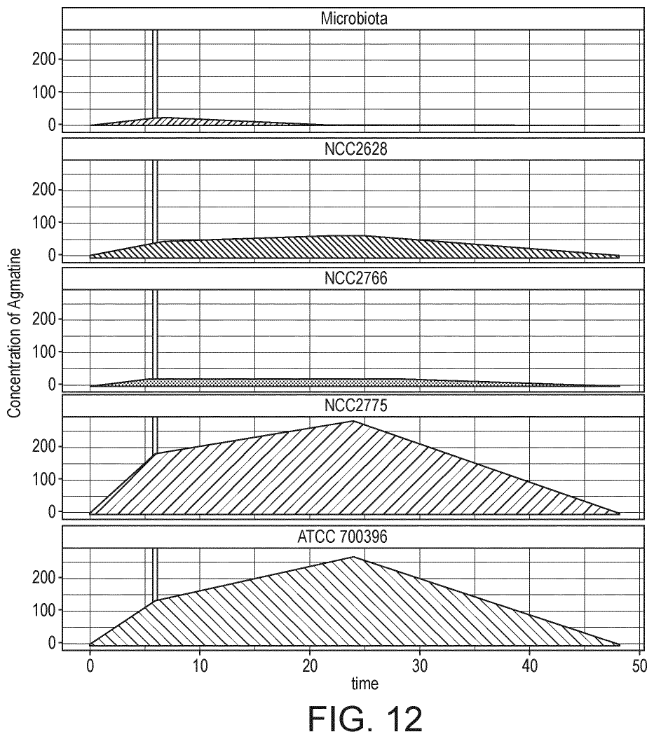

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the term “gut” refers to the organs, glands, tracts, and systems that are responsible for the transfer and digestion of food, absorption of nutrients, and excretion of waste. In humans, the gut comprises the gastrointestinal tract. The gut also comprises accessory organs and glands, such as the spleen, liver, gallbladder and pancreas. Bacteria can be found throughout the gut, e.g., in the gastrointestinal tract, and particularly in the intestines.

As used in this disclosure and the appended claims, the term “gastrointestinal tract” (also known as GI tract, GIT, digestive tract, digestion tract, or alimentary canal) refers to the tract from the mouth to the anus, which includes all the organs of the digestive system in humans and other animals. Food taken in through the mouth is digested to extract nutrients and absorb energy, and the waste expelled as feces. The gastrointestinal tract comprises the mouth, esophagus, stomach, small intestine, and large intestine. The human gastrointestinal tract includes the mouth, esophagus, stomach, and intestines, and is divided into the upper and lower gastrointestinal tracts. The GI tract includes all structures between the mouth and the anus, forming a continuous passageway that includes the main organs of digestion, namely, the stomach, small intestine, and large intestine. However, the complete human digestive system is made up of the gastrointestinal tract plus the accessory organs of digestion (the tongue, salivary glands, pancreas, liver and gallbladder).

As used in this disclosure and the appended claims, the term “upper gastrointestinal tract” refers to the gastrointestinal tract comprising the mouth, pharynx, esophagus, stomach, and duodenum of the small intestine.

As used in this disclosure and the appended claims, the term “lower gastrointestinal tract” refers the gastrointestinal tract comprising the remainder of the small intestine, i.e., the jejunum and ileum, and all of the large intestine, i.e., the cecum, colon, rectum, and anal canal. Bacteria can be found in the gastrointestinal tract, and particularly in the intestines.

As used in this disclosure and the appended claims, the term “non-pathogenic bacteria” refers to bacteria that are not capable of causing disease or harmful responses in a host. In an embodiment, non-pathogenic bacteria are commensal bacteria. Examples of non-pathogenic bacteria include, but are not limited to Bacillus, Bacteroides, Bifidobacterium, Brevibacteria, Clostridium, Escherichia coli, Lactobacillus (such as Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus), Lactococcus, Saccharomyces, and Staphylococcus. Naturally pathogenic bacteria may be genetically engineered to reduce or eliminate pathogenicity. A particular strain of bacteria can be nonpathogenic in one species but pathogenic in another. One species of bacterium can have many different types or strains. One strain of a bacterium species can be nonpathogenic and another strain of the same bacterium can be pathogenic.

As used in this disclosure and the appended claims, the term “probiotic” refers to live, non-pathogenic microorganisms, e.g., bacteria, which can confer health benefits to a host organism that contains an appropriate amount of the microorganism, generally by improving or restoring the gut flora or microbiota. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Bifidobacterium, Escherichia coli, Lactobacillus acidophilus, Lactobacillus bulgaricus, Lactobacillus paracasei, Lactobacillus plantarum, and Saccharomyces boulardii. The isolated probiotic may be a variant or a mutant strain of bacterium. Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used in this disclosure and the appended claims, the term “composition” refers to a preparation of a probiotic bacteria of the present invention with other components such as fats, proteins, starch, flavouring agents, vitamins, prebiotics, cellulose derivatives, gelatin, surfactants, polyethylene glycols, calcium bicarbonate, calcium phosphate, and dietary fibres.

All percentages expressed herein are by weight of the total weight of the composition unless expressed otherwise. When reference herein is made to the pH, values correspond to pH measured at about 25° C. with standard equipment.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but not preclude the presence of additional or further features.

Nevertheless, the compositions and methods disclosed herein may lack any element that is not specifically disclosed herein. Thus, a disclosure of an embodiment using the term “comprising” is (i) a disclosure of embodiments having the identified components or steps and also additional components or steps, (ii) a disclosure of embodiments “consisting essentially of” the identified components or steps, and (iii) a disclosure of embodiments “consisting of” the identified components or steps. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one of monobasic sodium phosphate or dibasic sodium phosphate” should be interpreted as “monobasic sodium phosphate,” or “dibasic sodium phosphate,” or “both monobasic sodium phosphate and dibasic sodium phosphate.”

Where used herein, the terms “example” and “such as,” particularly when followed by a listing of terms, are merely exemplary and illustrative and should not be deemed to be exclusive or comprehensive.

A “subject” or “individual” or “host organism” is a mammal, preferably a human. As used herein, an “effective amount” is an amount that prevents a deficiency, treats a disease or medical condition in an individual, or, more generally, reduces symptoms, manages progression of the disease, or provides a nutritional, physiological, or medical benefit to the individual.

The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

The term “unit dosage form”, as used herein, refers to physically discrete units suitable as unitary dosages for human and animal subjects, each unit containing a predetermined quantity of the composition disclosed herein in amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

The term “mM”, as used herein, refers to a molar concentration unit of an aqueous solution, which is mmol/L. For example, 1.0 mM equals 1.0 mmol/L. The term “μM”, as used herein, refers to a molar concentration unit of an aqueous solution, which is μmol/L. For example, 1.0 μM equals 1.0 μmol/L.

The terms “substantially no,” “essentially free” or “substantially free” as used in reference to a particular component means that any of the component present constitutes less than about 3.0% by weight, such as less than about 2.0% by weight, less than about 1.0% by weight, preferably less than about 0.5% by weight or, more preferably, less than about 0.1% by weight.

### EMBODIMENTS

The present disclosure generally relates to probiotics, compositions and methods for producing agmatine using microbiota, more particularly, relates to probiotics, compositions and methods for increasing production of agmatine and/or polyamines in the gastrointestinal tract of a subject in order to provide to the subject certain health benefits such as pain relief, antiaging effects, neuroprotective and antidepressant effects, improved vasodilatation and metabolic health, improved cellular health, and longevity. In an embodiment, the subject may be a mammal, preferably a human including adults and children. In an embodiment, the isolated probiotic is capable of colonizing and surviving in the gastrointestinal tract of the subject and capable of converting arginine to agmatine in the gastrointestinal tract of the subject. In an embodiment, the composition comprises a probiotic and arginine.

In an aspect, the present disclosure provides a probiotic capable of colonizing and surviving in a gastrointestinal tract of a subject, the isolated probiotic is capable of producing agmatine in the gastrointestinal tract of the subject using microbiota. In an embodiment, the isolated probiotic is capable of producing agmatine from arginine in the gastrointestinal tract or a lower gastrointestinal tract of the subject.

In an embodiment, the isolated probiotic is a bacterial strain capable of colonizing and surviving in the gastrointestinal tract of the subject, and capable of converting arginine to agmatine and/or polyamines such as putrescine, spermidine and spermine at the gastrointestinal tract of the subject. In an embodiment, the isolated probiotic is a bacterial strain capable of converting arginine to agmatine and/or polyamines such as putrescine, spermidine and spermine using arginine decarboxylase (ADC). In an embodiment, the isolated probiotic is a bacterial strain capable of converting arginine to agmatine and/or polyamines such as putrescine, spermidine and spermine using ornithine decarboxylase (ODC).

In an embodiment, the isolated probiotic is a bacterial strain capable of colonizing and surviving in the gastrointestinal tract of the subject. In an embodiment, the isolated probiotic is capable of converting arginine to agmatine in the gastrointestinal tract of the subject. In an embodiment, the isolated probiotic is a bacterial strain capable of converting arginine to agmatine using arginine decarboxylase (ADC). In an embodiment, the isolated probiotic is a bacterial strain capable of boosting the production of arginine decarboxylase (ADC) and converting arginine to agmatine using ADC in the gastrointestinal tract of the subject.

In an embodiment, the isolated probiotic is Lactobacillus acidophilus. In an embodiment, the Lactobacillus acidophilus is capable of converting arginine to agmatine using arginine decarboxylase (ADC). In an embodiment, the Lactobacillus acidophilus is a strain comprises the endogenous enzyme arginine decarboxylase (ADC). In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396).

In an embodiment, the isolated probiotic is a bacterial strain having at least 90%, 91%, 92%, 93%, 94%, 95%, 96%, 97%, 98%, 99%, 99.5% or 99.9% sequence identity to one of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396).

In an embodiment, the isolated probiotic is a Lactobacillus acidophilus strain has at least 90%, 91%, 92%, 93%, 94%, 95%, 96%, 97%, 98%, 99%, 99.5% sequence identity to one of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396).

In an embodiment, the isolated probiotic is capable of converting arginine to agmatine in the gastrointestinal tract of the subject at the pH of at least about 4.0, at least about 5.0, at least about 6.0, between about 5.0 and about 9.0, between about 6.0 and about 8.0, or about 7.0.

In an embodiment, the isolated probiotic is capable of converting arginine to agmatine in the gastrointestinal tract of the subject in the presence of a prebiotic such as fructooligosaccharide (FOS).

In an embodiment, the isolated probiotic is capable of converting arginine to agmatine in the gastrointestinal tract of the subject to achieve an agmatine concentration of at least 5 μM, at least 10 μM, at least 20 μM, at least 30 μM, at least 40 μM, at least 50 μM, at least 60 μM, at least 70 μM, at least 80 μM, at least 90 μM, at least 95 μM, at least 100 μM, at least 105 μM, at least 110 μM, at least 115 μM, at least 120 μM, at least 125 μM, at least 130 μM, at least 200 μM; at least 300 μM, at least 400 μM, at least 500 μM; at least 600 μM, at least 700 μM, or at least 800 μM at 24 hours after administering the composition.

In an aspect, the present disclosure provides a composition for increasing the production of agmatine and/or polyamines in a body part of a subject using microbiota in order to provide to the subject certain health benefits such as pain relief, antiaging effects, neuroprotective and antidepressant effects, improved vasodilatation and metabolic health, improved cellular health, and longevity.

In an embodiment, the present disclosure provides a composition for improving pain relief, antiaging effects, neuroprotective and antidepressant effects, vasodilatation and metabolic health, cellular health, and longevity in a subject in need thereof. The composition comprises a microorganism or bacteria. In an embodiment, the microorganism is a probiotic. In an embodiment, the isolated probiotic is capable of increasing the production of agmatine and/or polyamines in a body part of the subject. In an embodiment, the isolated probiotic is capable of colonizing and surviving in the gastrointestinal tract of the subject.

In an embodiment, the body part of the subject is the gastrointestinal tract, a lower gastrointestinal tract, an intestine, a small intestine or a large intestine of the subject. In an embodiment, the production of agmatine and/or polyamines is at the gastrointestinal tract of the subject. In an embodiment, the production of agmatine is at the lower gastrointestinal tract of the subject. In an embodiment, the production of agmatine is at the small intestine of the subject.

In an embodiment, the production of agmatine is at the large intestine of the subject. In an embodiment, the isolated probiotic is capable of colonizing and surviving in the gastrointestinal tract of the subject, and further capable of increasing the production of agmatine in gastrointestinal tract of the subject.

In an embodiment, the composition further comprises arginine. In an embodiment, the isolated probiotic is a bacterial strain capable of converting arginine to agmatine and/or polyamines such as putrescine, spermidine and spermine at the gastrointestinal tract of the subject. In an embodiment, the isolated probiotic is a bacterial strain capable of converting arginine to agmatine and/or polyamines such as putrescine, spermidine and spermine using arginine decarboxylase (ADC). In an embodiment, the isolated probiotic is a bacterial strain capable of converting arginine to agmatine and/or polyamines such as putrescine, spermidine and spermine using omithine decarboxylase (ODC). In an embodiment, the isolated probiotic is a bacterial strain capable of boosting the production of arginine decarboxylase (ADC) and converting arginine to agmatine using ADC in the gastrointestinal tract of the subject.

In an embodiment, the isolated probiotic is Lactobacillus acidophilus. In an embodiment, the composition comprises Lactobacillus acidophilus and arginine. In an embodiment, the Lactobacillus acidophilus is capable of converting arginine to agmatine and/or other polyamines such as putrescine, spermidine and spermine using arginine decarboxylase (ADC). In an embodiment, the Lactobacillus acidophilus is a strain comprises the endogenous enzyme arginine decarboxylase (ADC).

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic is a bacterial strain having at least 90%, 91%, 92%, 93%, 94%, 95%, 96%, 97%, 98%, 99%, 99.5% or 99.9% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain has at least 90%, 91%, 92%, 93%, 94%, 95%, 96%, 97%, 98%, 99%, 99.5% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the composition further comprises an acidifying compound. In an embodiment, the acidifying compound is a prebiotic such as fructooligosaccharide (FOS). In an embodiment, the composition further comprises a prebiotic. In an embodiment, the composition further comprises fructooligosaccharide (FOS).

In an embodiment, the composition further comprises a co-factor such as pyridoxal-5′-phosphate (PLP).

In an embodiment, the concentration of the agmatine in the gastrointestinal tract of the subject is at least 5 μM, at least 10 μM, at least 20 μM, at least 30 μM, at least 40 μM, at least 50 μM, at least 60 μM, at least 70 μM, at least 80 μM, at least 90 μM, at least 95 μM, at least 100 μM, at least 105 μM, at least 110 μM, at least 115 μM, at least 120 μM, at least 125 μM, at least 130 μM, at least 200 μM; at least 300 μM, at least 400 μM, at least 500 μM; at least 600 μM, at least 700 μM, or at least 800 μM at 24 hours after administering the composition.

In an embodiment, the composition further comprises one or more of a starch source, a protein source and lipid source.

Suitable starch sources are, for example, grains and legumes such as corn, rice, wheat, barley, oats, soy, and mixtures of these.

Suitable protein sources may be selected from any suitable animal or vegetable protein source, for example meat and meal, poultry meat or meal, fish meat or meal, soy protein concentrates, milk proteins, gluten, and the like.

Suitable lipid sources include meats, animal fats and vegetable oils or fats.

The choice of the starch, protein and lipid sources will be largely determined by the nutritional needs of the subject, palatability considerations, and the type of product applied.

Further, various other ingredients, for example, sugar, salt, spices, seasonings, vitamins, minerals, flavoring agents, fats and the like may also be incorporated into the composition as desired.

In an embodiment, the composition is in a form of a dried powder, a capsule, a shelf stable liquid, or a wet, chilled or shelf stable paste. In an embodiment, the composition is a powder. In an embodiment, the composition is in a form of a capsule. In an embodiment, the composition is a dried powder and the isolated probiotic bacteria is coated onto or filled into the composition. In an embodiment, the isolated probiotic bacterial is active in the final composition.

In an embodiment, the isolated probiotic is capable of surviving the gastric acid environment of the subject for at least 10 minutes, at least 20 minutes, at least 30 minutes, at least 40 minutes, at least 50 minutes, at least 60 minutes, or at least 70 minutes.

In an embodiment, the isolated probiotic is capable of surviving in a pH 1.5 environment for at least 10 minutes.

In an embodiment, the isolated probiotic is capable of surviving in a pH 2.6 environment for at least 10 minutes.

In an embodiment, the isolated probiotic is capable of surviving in a pH 3.4 environment for at least 60 minutes.

In an embodiment, the subject is a human. The gastrointestinal pH profile of a healthy subject is described below. The intraluminal pH is rapidly changed from highly acid in the stomach to about pH 6 in the duodenum. The pH gradually increases in the small intestine from pH 6 to about pH 7.4 in the terminal ileum. The pH drops to 5.7 in the caecum, but again gradually increases, reaching pH 6.7 in the rectum. In order for the isolated probiotic to reach the lower gastrointestinal tract to colonizing and converting arginine to agmatine, the isolated probiotic needs to survive the highly acidic fluid environment in the stomach either by itself or by being protected in a capsule which can tolerate the highly acidic fluid environment in the stomach.

The normal volume of the human stomach fluid is about 20 to about 100 mL and the stomach fluid is highly acidic, also known as a gastric acid. The gastric acid in the human stomach lumen typically has a pH of about 1.5 to 3.5, a level maintained by the proton pump H+/K+ ATPase. The highly acidic environment in the stomach lumen degrades food including proteins. The parietal cell releases bicarbonate into the bloodstream in the process, which causes a temporary rise of pH in the blood, known as an alkaline tide. When a meal is ingested, gastric pH increases due to the buffering effect of the meal and then returns to baseline due to secretion of gastric acid.

In the present disclosure, the isolated probiotic is capable of surviving the gastric acid environment of the human stomach to reach the small intestine, the large intestine or the lower gastrointestinal tract where the isolated probiotic colonize and convert arginine to agmatine using arginine decarboxylase (ADC) and the local microbiota.

In the present disclosure, different probiotic strains were screened to identify the strains that were stable when incubated in incubation solutions mimicking the gastric acid of the human stomach fluid. The strains tested in the present disclosure including Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396) were stable in the incubation solutions for at least 10 minutes, at least 20 minutes, or at least 30 minutes at pH of about 1.5; and at least 10 minutes, at least 20 minutes, or at least 30 minutes at pH of about 2.6; and at least 60 minutes or at least 70 minutes at a pH of about 3.4.

In an embodiment, the isolated probiotic is capable of surviving the gastric acid environment of the human stomach for at least 10 minutes, at least 20 minutes, at least 30 minutes, at least 40 minutes, at least 50 minutes, at least 60 minutes, or at least 70 minutes.

In an embodiment, the composition is in a form of a capsule. In an embodiment, the isolated probiotic in the capsule is capable of surviving the gastric acid environment of the human stomach for at least 10 minutes, at least 20 minutes, at least 30 minutes, at least 40 minutes, at least 50 minutes, at least 60 minutes, or at least 70 minutes.

In an embodiment, the capsule enables the isolated probiotic to survive the gastric acid environment of the human stomach for at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 2 hours, at least 3 hours, or at least 4 hours.

In an aspect, the present disclosure provides a method for increasing the production of agmatine and/or polyamines in a body part of a subject using microbiota in order to provide to the subject certain health benefits such as pain relief, antiaging effects, neuroprotective and antidepressant effects, improved vasodilatation and metabolic health, improved cellular health, and longevity. The method comprising administering to the subject a composition.

In an aspect, the present disclosure provides a method improving pain relief, antiaging effects, neuroprotective and antidepressant effects, improved vasodilatation and metabolic health, improved cellular health, and longevity in need thereof by increasing the production of agmatine and/or polyamines in a body part of a subject using microbiota, the method comprising administer to the subject a composition discussed herein above and elsewhere in the present disclosure.

In an embodiment, the method further comprises administering the composition in an effective amount to the subject to provide at least 500-700 mg arginine per day and/or at least 5-7 million CFU of the isolated probiotic per day for at least 1 month, at least 3 months, or at least 6 months.

In an embodiment, the composition comprises a probiotic capable of colonizing and surviving in the gastrointestinal tract of the subject, and further capable of increasing the production of agmatine in gastrointestinal tract of the subject.

In an embodiment, the composition further comprises arginine.

In an embodiment, the composition further comprises a prebiotic. In an embodiment, the prebiotic is fructooligosaccharide (FOS) or galactooligosaccharide (GOS).

In an embodiment, the composition further comprises a co-factor such as pyridoxal-5′-phosphate (PLP).

In an embodiment, the method further comprising administering an acidifying compound such as a prebiotic separately to the subject before or after administering the composition to adjust the pH value of the GIT. In an embodiment, the acidifying compound is FOS.

In an embodiment, the body part of the subject is the gastrointestinal tract of the subject. In an embodiment, the production of agmatine and/or polyamines is at the gastrointestinal tract of the subject. In an embodiment, the production of agmatine is at the lower gastrointestinal tract of the subject. In an embodiment, the production of agmatine is at the lower gastrointestinal tract of the subject. In an embodiment, the production of agmatine is at the small intestine of the subject. In an embodiment, the production of agmatine is at the large intestine of the subject.

In an embodiment, the isolated probiotic is a bacterial strain capable of converting arginine to agmatine and/or polyamines such as putrescine, spermidine and spermine at the gastrointestinal tract of the subject. In an embodiment, the isolated probiotic is a bacterial strain capable of converting arginine to agmatine and/or polyamines such as putrescine, spermidine and spermine using arginine decarboxylase (ADC). In an embodiment, the isolated probiotic is a bacterial strain capable of converting arginine to agmatine and/or polyamines such as putrescine, spermidine and spermine using ornithine decarboxylase (ODC). In an embodiment, the isolated probiotic is a bacterial strain capable of boosting the production of arginine decarboxylase (ADC) and converting arginine to agmatine using ADC in the gastrointestinal tract of the subject.

In an embodiment, the isolated probiotic is Lactobacillus acidophilus. In an embodiment, the composition comprises Lactobacillus acidophilus and arginine. In an embodiment, the Lactobacillus acidophilus is capable of converting arginine to agmatine and/or other polyamines such as putrescine, spermidine and spermine using arginine decarboxylase (ADC). In an embodiment, the Lactobacillus acidophilus is a strain comprises the endogenous enzyme arginine decarboxylase (ADC).

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic is a bacterial strain having at least 90%, 91%, 92%, 93%, 94%, 95%, 96%, 97%, 98%, 99%, 99.5% or 99.9% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain has at least 90%, 91%, 92%, 93%, 94%, 95%, 96%, 97%, 98%, 99%, 99.5% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the composition is in a form of a dried powder, a capsule, a shelf stable liquid, or a wet, chilled or shelf stable paste. In an embodiment, the composition is a powder. In an embodiment, the composition is in a form of a capsule. In an embodiment, the composition is a dried powder and the isolated probiotic is coated onto or filled into the composition. In an embodiment, the isolated probiotic bacterial is active in the composition.

In an embodiment, the concentration of the agmatine in the gastrointestinal tract of the subject is at least 5 μM, at least 10 μM, at least 20 μM, at least 30 μM, at least 40 μM, at least 50 μM, at least 60 μM, at least 70 μM, at least 80 μM, at least 90 μM, at least 95 μM, at least 100 μM, at least 105 μM, at least 110 μM, at least 115 μM, at least 120 μM, at least 125 μM, or at least 130 μM at about 24 hours after administering the composition.

In an embodiment, the subject may be a mammal, preferably a human including adults and children.

In an aspect, the present disclosure provides an isolated probiotic capable of colonizing and surviving in a gastrointestinal tract of a subject, wherein the isolated probiotic is capable of producing agmatine from arginine in the gastrointestinal tract of the subject using microbiota.

In an embodiment, the isolated probiotic is capable of producing agmatine from arginine in the gastrointestinal tract of the subject using microbiota further using arginine decarboxylase (ADC), and the isolated probiotic is bacterial strain selected from a group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the isolated probiotic is a bacterial strain having at least 90%, preferably at least 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain that has at least 90%, preferably at least 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is capable of producing agmatine from arginine in the gastrointestinal tract of the subject using microbiota at a pH between about 4.0 and about 8.0.

In an embodiment, the isolated probiotic is capable of producing agmatine from arginine in the gastrointestinal tract of the subject using microbiota in the presence of fructooligosaccharide (FOS).

In an embodiment, the isolated probiotic is capable of producing agmatine from arginine in the gastrointestinal tract of the subject using microbiota and arginine decarboxylase (ADC) in the presence of co-factor pyridoxal-5′-phosphate (PLP).

In an embodiment, the isolated probiotic has the capability of increasing the bioavailability of Agmatine, produced from arginine in the gastrointestinal tract of the subject, through delaying the transformation of Agmatine to downstream polyamines, by at least 24 hours.

In an embodiment, the isolated probiotic is capable of producing agmatine from arginine in the gastrointestinal tract of the subject using microbiota and arginine decarboxylase (ADC), wherein the concentration of the agmatine is at least 20 μM at 24 hours after administering the isolated probiotic.

In an embodiment, the isolated probiotic is capable of surviving in a pH 2.6 environment for at least 10 minutes.

In an embodiment, the isolated probiotic is capable of surviving in a pH 3.4 environment for at least 60 minutes.

In an embodiment, the gastrointestinal tract of the subject is a lower gastrointestinal tract of the subject.

In an embodiment, the gastrointestinal tract of the subject is large intestine of the subject.

In an aspect, the present disclosure provides a composition for increasing production of agmatine in a gastrointestinal tract of a subject using microbiota to provide to the subject health benefits including pain relief, antiaging effects, neuroprotective and antidepressant effects, decreased age-related memory loss, improved vasodilatation and metabolic health, improved cellular health, and longevity, the composition comprising: an isolated probiotic; and arginine, wherein the isolated probiotic is capable of colonizing and surviving in the gastrointestinal tract of the subject and capable of producing agmatine from arginine in the gastrointestinal tract of the subject using microbiota.

In an embodiment, the composition further comprises fructooligosaccharide (FOS) and/or GOS.

In an embodiment, the composition further comprises a co-factor pyridoxal-5′-phosphate (PLP).

In an embodiment, the composition further comprises one or more of a starch source, a protein source, a prebiotic source, lipid source, vitamins, sugars, salt, spices, seasonings, minerals, and flavoring agents.

In an embodiment, the composition is in a form of a capsule.

In an embodiment, the composition is in a form of a dried powder, and the isolated probiotic is filled into the composition.

In an embodiment, the isolated probiotic is active in the composition.

In an embodiment, the isolated probiotic is a Lactobacillus acidophilus strain that has at least 90%, preferably at least 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an aspect, the present disclosure provides a method for increasing production of agmatine in a gastrointestinal tract of a subject using microbiota to provide to the subject health benefits including pain relief, antiaging effects, neuroprotective and antidepressant effects, decreased age-related memory loss, improved vasodilatation and metabolic health, improved cellular health, and longevity, the method comprising: administering to the subject a composition of claim 13, the composition comprising: an isolated probiotic; and arginine, wherein the isolated probiotic is capable of colonizing and surviving in the gastrointestinal tract of the subject and capable of producing agmatine from arginine in the gastrointestinal tract of the subject using microbiota.

In an embodiment, the composition is administered to the subject in an effective amount to provide the subject a daily dose of the isolated probiotic in a range of 5M-10B CFU and a daily dose of the arginine in a range of 500-750 mg.

In an embodiment, the isolated probiotic is a Lactobacillus acidophilus strain at least 90%, preferably has at least 95% sequence identity to one or more of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

In an embodiment, the isolated probiotic is a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

In an embodiment, the subject is a human.

In an embodiment, a concentration of the agmatine in the gastrointestinal tract of the subject is at least 20 μM at about 24 hours after administering the composition.

In an aspect, the present disclosure provides a method for improving pain relief, antiaging effects, neuroprotective and antidepressant effects, improved vasodilatation and metabolic health, improved cellular health, and longevity of a subject in need thereof by increasing production of agmatine in a gastrointestinal tract of the subject using microbiota, the method comprising administer to the subject the composition of claim 13.

Agmatine produced by decarboxylation of arginine, is one of the precursors of the polyamines such as putrescine, spermidine and spermine in plant, prokaryotes and some mammalian cells. Putrescine, spermidine, spermine are essential for normal cell growth and viability. Spermidine is a cytoprotective and an autophagy inducer, its supplementation has been linked with antiaging effects in preclinical and clinical experiments[1, 2]. Higher systemic and urinary polyamines level are linked with growth in healthy children, but in the context of cancer, these molecules may also be associated with tumor progression[3, 4].

Besides being an intermediate of polyamines production, agmatine induces a variety of physiological and pharmacological effects on the central nervous system and other organs[5, 6]. Several health benefits have been attributed to the supplementation of synthetic agmatine. The most described and substantiated benefits are agmatine neuroprotective and antidepressant effects which are supported by several pre-clinical studies using animal model of brain ischemia, hypoxia, drug-based toxicity or behavioral test predicting antidepressant activity (tail suspension and forced swim test) [7, 8]. The effect of agmatine supplementation on pain release is also well substantiated with a clinical trial on patients suffering from radiculopathy and several pre-clinical studies[6, 7]. Early evidence (based on in-vitro or ex-vivo experiments) are found on the role of agmatine on vasodilatation, improved metabolic health (stimulation of fatty acid oxidation, decreased lipid peroxidation, improved insulin signalling) and cellular health (reduction of oxidative stress, cytoprotection, pro and antiproliferative effect) [5, 6].

The mechanism underlying the reported benefits of agmatine involved non receptors and receptors-based effects[5]. Agmatine activates α-2 adrenoreceptors and imidazoline receptors at high affinity (Ki 0.8-164 μM for α-2 adrenoreceptors or 0.33 to >300 μM for imidazoline receptors)[5]. Those trigger secondary messengers involved in anti-inflammatory, nitric oxide regulation, Ca²⁺ regulation, antioxidation and cell survival in different tissues[9-11]. These receptors are distributed in several organs including brain (the most reported site of action), gastrointestinal (GI) tract and liver[5]. Importantly these receptors are not activated by spermidine, spermine, putrescine or arginine at physiological level[12].

Agmatine, putrescine, spermidine and spermine are also naturally found in a large variety of food under a free or conjugated form (for instance Phenol amides)[13]. A significant proportion of the systemic polyamines and agmatine may come from dietary origin, however the exact contribution is not known[14]. It is even more difficult to evaluate the contribution of dietary agmatine as agmatine daily consumption has not been established to the extent of our knowledge.

Free form of dietary polyamines and agmatine are quickly and effectively absorbed in the upper GI tract mainly in the stomach, duodenum and jejunum[15]. Limited recovery of agmatine and putrescine in the blood after dietary intervention with radiolabeled molecules suggests that polyamines are rapidly absorbed and metabolized in the intestinal wall, liver and/or other organs[16, 17]. Dietary agmatine is mostly accumulated in the liver and stomach wall[15]. During fasting, important gut luminal polyamine production and turnover are still found may be due to microbial activity, pancreatic secretions, intestinal death cells and mucosal production[18]. Overall, the order of magnitude for polyamines in the gut lumen is putrescine>spermidine>spermine[17]. No information has been found on gut luminal agmatine concentration.

Polyamines can also be produced by the gut microbiome and contrary to most mammalian cells where omithine decarboxylase (ODC) is the rate limiting enzyme, the primarily synthesis route is thought to be via arginine decarboxylase (ADC) and agmatine production[17]. Considering the effective absorption of dietary polyamines in the upper GI tract, the presence of putrescine, spermidine, spermine and agmatine in the ileum, caecum and colon is mostly attributed to microbial activity[17]. A novel hybrid mechanism for polyamine production has been described and requires the collaboration of several gut bacterial taxa groups through agmatine cross-feeding[19]. The presence of arginine, the optimization of ADC activity and acid production have been suggested as key factors for boosting agmatine and polyamines production through this new hybrid mechanism[19].

Gut luminal polyamines undergo entero-hepatic circulation[20]. However, polyamines absorption in caecum and colon is different from those in duodenum and jejunum as limited part of the molecules are taken up in the pancreato-biliary fluid circulation[17]. It is unclear how the microbiome-derived polyamines are absorbed, redistributed and metabolized in the host. There is no information on the exact contribution of microbial polyamines and agmatine on the overall molecular pool. Additional pre-clinical experiments with enema administration of polyamines would be required to clarify this point. Luminal polyamines and agmatine can also be absorbed by intestinal cells via active transporters where they can confer local effects or be exported further to the host [21].

Limited data in rodents showed that agmatine is the most abundant in stomach, intestinal and liver tissues[20]. It is found in very low concentration in brain, although it is the most reported sight of action[22]. It looks like agmatine is 10 to 100 time less abundant than spermine or spermidine in rat tissues, however this comparison needs to be taken with cautious as different rat species and analytical methods have been used among publications[23]. In human urine and serum, agmatine, spermidine and spermine are found in the same range of concentration (around 0.5 μM) [24]. The amount of agmatine in the human distal gut is unknown but limited data show that fecal agmatine content is slightly higher (˜10 μmol/g dry matter) than putrescine or spermidine suggesting that the gut microbiome may produce agmatine at relatively high concentration and could excess a local effect at least in the gut [24]. Another study shows that fecal putrescine is significantly higher than spermidine and can reach around 700 μM in healthy adults [25]. Agmatine concentration is not reported in that study [25]. However, due to the lack of large and quantitative data in healthy adult population, it is difficult to draw conclusion on the distribution of agmatine and polyamines in human biofluids. Polyamines and agmatine are quickly reabsorbed and metabolized after dietary or intravenous (IV) administration (half-life of agmatine after IV injection is 5 minutes), suggesting that monitoring systemic polyamine level may not be the best way to follow an increase in gut luminal polyamines[26, 27]. Acetylated spermine and spermidine may be better candidates to follow the changes in the polyamine pathway[28].

Boosting the microbiome-derived polyamine pathway may have a beneficial effect on the host, although the exact mechanism of interaction between the intestinal polyamines and the sight of action remain unknown [29, 30]. For instance, the combination of arginine and bifidobacteria LKM512 in mouse model is found to increase gut microbial putrescine and spermidine by comparison to arginine, the isolated probiotic alone or the control, and the effect is abolished after antibiotics treatment. Six-month supplementation with this double treatment improves longevity and decrease age-related memory loss in the same mouse model by comparison to the single treatment or the control [31]. In a recent study in osteoporosis mouse model, Chevalier et al demonstrate that the beneficial effect of warm exposure on bone strength is mediated by changes in the microbiome composition, functionalities and an increase in the bacterial synthetic polyamine pathway[32].

The present disclosure discloses a composition, such as a composition in a form of a capsule or a powder, to naturally increase agmatine and/or polyamines by boosting the gut microbiome production. Applicant has tested the following parameters using available in-vitro gut models: 1) arginine source, the precursor of agmatine and polyamines; 2) gut environmental acidification; and 3) addition of probiotics and/or cofactors to boost ADC and/or agmatine production.

Applicant has surprisingly discovered that increasing these metabolites such as agmatine and polyamines may induce a local effect in the gut and potentially a systemic effect.

### Estimated Concentration of Gut-Derived Agmatine and Spermidine

The effective doses of polyamines and agmatine for a specific benefit and the toxic doses are determined based on clinical and pre-clinical experiments with synthetic molecules[33]. By calculating a rough concentration of human fecal and ileal polyamines, Applicant estimated that the gut luminal polyamines content is lower than the required synthetic dose for a benefit or a toxic dose. However, the estimated fecal agmatine and spermidine are above the dose required for a mechanistic effect (based on receptors affinity or in-vitro autophagy dose-response experiments). For instance, agmatine can activate α-2 adrenoreceptors and imidazoline receptors triggering a mechanism of action at low concentration in the brain, stomach or platelet membranes. Since G protein-coupled receptors (GPCRs) are also present in the gut, a local increase in agmatine in ileum and colon may be enough to activate these receptors. The spermidine and agmatine concentrations in feces and in-vitro batch fermentations and comparison with concentrations required for a biological effect (based on in-vitro assays) are listed in Table 1 below.

TABLE 1
Spermidine and agmatine concentration in feces, in-vitro
batch fermentations and comparison with concentration required
for a biological effect (based on in-vitro assays).

| | Spermidine | | Agmatine | |
|---|---|---|---|---|
| Feces (μmol/g of dry matter) | ~2.0*-25** | | ~10* | |
| (*: [24] **: [25]) | | | | |
| Estimated Fecal concentration in | 666 | μM | 3333 | μM |
| wet feces (75% water) based on | | | | |
| [24] | | | | |
| Estimated concentration in ileum | 5.75 | μM | — | |
| from 20 min collection[18] | | | | |
| Affinity for α-2 adrenoreceptors | — | | 0.8-164 | μM |
| (Ki) | | | | |
| Affinity for imidazoline receptors | — | | 0.33 to >300 μM | |
| (Ki) | | | (depend on organs) | |
| Dose required for cytoprotective | 10-100 μM | | — | |
| effect/e.g. autophagy | (depending on | | | |
| | organ/cell type) | | | |
| Average concentration in Prodigest | 4.38 | μM | 0.97 | μM |
| exp (fermentation with arginine | | | | |
| alone) | | | | |
| Average Concentration in house | 11.95 | μM | 35.29 | μM |
| batch (fermentation with arginine | | | | |
| alone) | | | | |

It is difficult to translate the polyamine production from the in-vitro gut models of the present disclosure with gut polyamine content in which production, absorption and metabolism occurs. The ranges of spermidine and agmatine concentrations from the in-vitro batch fermentation experiments are lower but in the same order of magnitude (μM) as those found in feces. The average agmatine and spermidine concentrations after arginine fermentation are already within the range of concentrations required for a mechanistic effect. However, the accurate concentrations to activate the receptors or triggering an autophagy effect in the gut are not known. Additional, in-vitro experiment results helped set up the target concentrations and leverage the local effect on microbiome-derived polyamines. Meanwhile it is difficult to estimate a target concentration of agmatine and spermidine for the ingredient selection of the present disclosure.

### Ingredient Selection Criteria

Ingredients are selected to provide the highest and the most significant increase in agmatine and spermidine production while limiting the production of putrescine which has been linked in some cases with the cancer progression. Evaluate synergetic effect between combination of ingredients are evaluated, and the ingredients that improve product differentiation and communication are considered.

### Further Design of Additional Analyses

Applicant has designed MiniGut experiments to: 1) confirm the agmatine-derived activation of GPCR receptors in healthy gut cells; 2) identify a target concentration for a local dose; and 3) provide first evidence on local effect of agmatine on gut health (i.e., cytoproliferation, anti-inflammation and anti-oxidation).

Applicant has designed Zebrafish and in-vitro autophagy experiments to: 1) support early evidence of agmatine on anti-ageing via autophagy activation; 2) compare agmatine effect with spermidine; and 3) identify the agmatine target concentration required for autophagy effects

Applicant has designed immune based assays.

Applicant has further conducted investigation of the CALM cohort to: 1) understand the link between microbial polyamines and agmatine and i) gut and ii) immune health in a population of free-living seniors; and 2) understand how the amounts of microbial polyamines and agmatine are affected by specific protein enriched supplements (whey or collagen proteins) in free-living seniors.

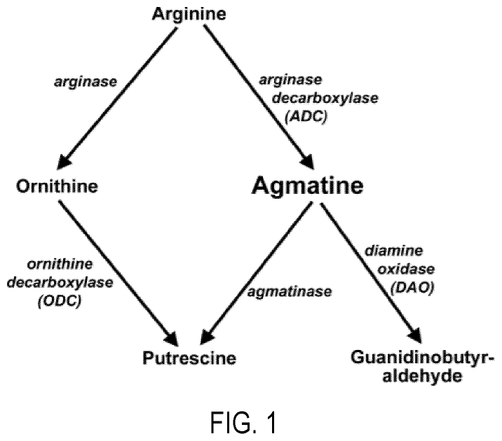

Agmatine is an intermediate of the polyamine's pathways. Agmatine is a biogenic amine produced by decarboxylation of arginine. This reaction is performed by arginine decarboxylase (ADC) which require pyridoxal-5′-phosphate (PLP) as cofactor. Agmatine is then distributed into two main pathways. It is converted into putrescine via agmatinase enzyme which is a precursor of the other polyamines spermidine and spermine; or it is transformed into guanidinobutyraldehyde via the amine oxidase or diamine oxidase (DAO)[34]. The substrate and enzymes involved in agmatine homeostasis is illustrated in FIG. 1. [34]

### The Different Sources of Agmatine and Polyamines

### Diet:

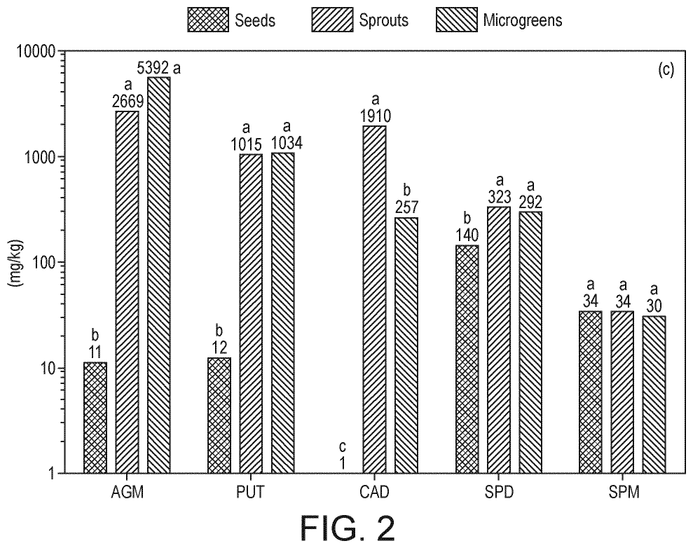

Polyamines are found in a large variety of food. The total polyamines daily intake (putrescine, spermidine and spermine) in the European Union is 353.6 μmol/day diet, putrescine being the most abundant (211.9 μmol/day diet) followed by spermidine (87 μmol/day diet)[35]. High agmatine concentration is found in fermented foods like alcoholic beverages (sake: 114 mg/L), sauerkraut brine (12 mg/L) and various Seeds (lentil: 38 mg/kg, Alfafa fenugreek: 11 mg/kg, Daikon radish: 52 mg/kg)[14]. No agmatine daily intake has been reported[14]. Seed germination leads to an increase in agmatine and other polyamines[36]. FIG. 2 illustrates the content of the agmatine (AGM), the polyamines such as putrescine (PUT), cadaverine (CAD), spermidine (SPD), spermine (SPM) in seeds, sprouts and microgreens of alfalfa. [36]

Phenolamides defined as polyamines conjugated with phenolic compounds are highly abundant in some plants and can be considered as a source of polyamines. Agmatine conjugated phenolamides are found in wheat, rice, maize, sozabean; while Hordatines (dimers of agmatine conjugated phenolamides) are particularly present in barely. However, bioavailability and digestibility of phenolamides needs to be further investigated[13, 37].

### Synthetic Form:

Spermidine, agmatine and arginine (the precursor of these polyamines) are also available as supplement products (powder or capsule) to support a range of benefits. The communicated benefits are supported by various level of evidences. The arginine or related polyamines in supplement products are listed in Table 2 below.

TABLE 2
Arginine or related polyamines in supplement product

| | Recommended daily | Communicated benefits |
|---|---|---|
| Molecules | dose on product | on product |
| Agmatine | 500-1000 mg (3.8-7 mmol) | Sport strength, mood, focus, |
| | | pain release |
| Spermidine | ~ 1 mg* (~6.6 μmol) | Cell renewal, longevity |
| Spermine | NA** | NA |
| Putrescine | NA** | NA |
| Arginine | 750-3000 mg | Vasodilatation effect, |
| | | sexual health |

*These supplements are often plant-based extracts containing spermidine; and
**No commercial product found

### Endogenous Production:

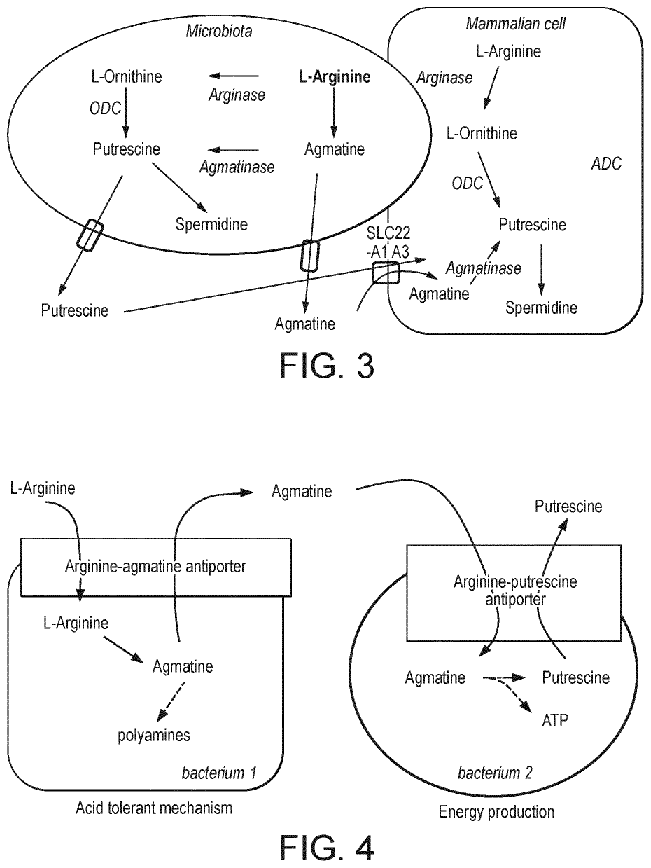

Polyamines in mammals are mainly produced via the arginase decarboxylase (ADC) and ornithine decarboxylase (ODC) route, as shown in FIG. 3. Nevertheless, ADC expression and agmatine production have been found in several mammalian cells including brain (glial cells, medulla), liver, and kidney[38, 39]. ADC is more often expressed in the mitochondria than cytoplasm. The ADC distribution in mammalian organs differ from those of agmatine suggesting an extracellular agmatine uptake[34, 40]. FIG. 3 illustrates the polyamine metabolism and transport in mammalian cells and microbiota.

As agmatine is positively charged at physiological pH, it cannot cross the cellular lipid barrier by simple diffusion. Instead, agmatine uptake is mediated by active polyamine transports system comprising the solute transport family (SLC) that contains about 400 annotated members as well as the organic cation family (OCT)[8, 40]. Active agmatine efflux has been identified in human glioma cells [22], rat hepatocytes [32], rat arterial smooth muscle cells [33], and hamster kidney cells [34]. Studies carried out on 6 cell lines of human intestinal origin (Caco2, Cx1, Colo320, HT29, Colo205E, SW480) shows an active agmatine uptake via agmatine-specific organic cations transporters[21, 41].

### Gut Microbiome:

Prokaryotes can produce polyamines via ODC or ADC (as shown in FIG. 3), however, ADC is thought to be the dominant route for polyamines synthesis by the gut microbiome[40, 42]. ADC has been characterized among several gut bacterial genera highlighting the capacity of the gut microbiome to produce agmatine[34].

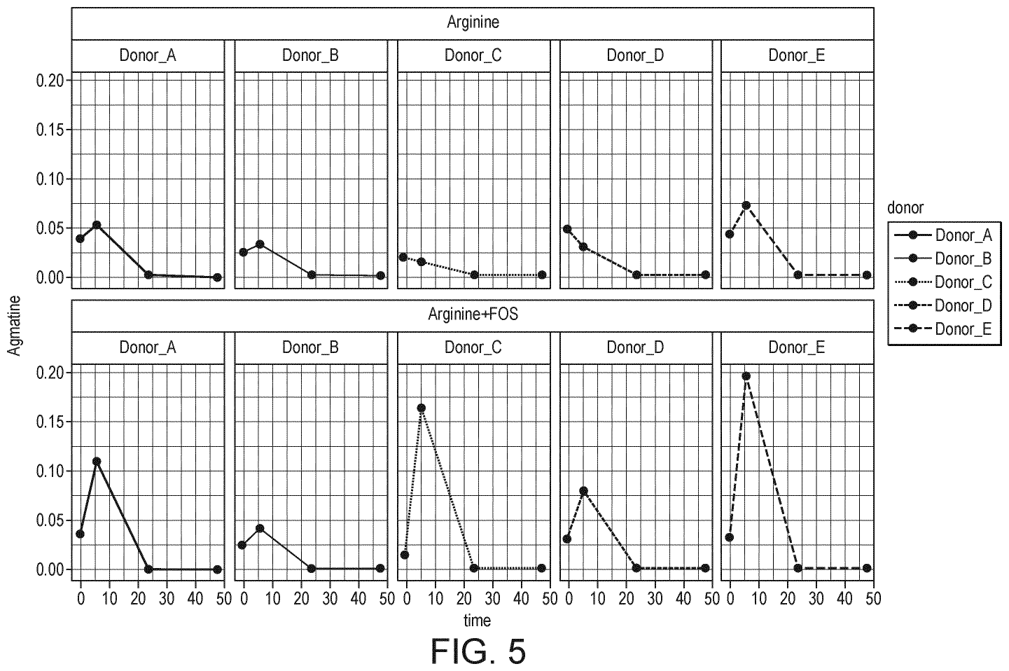

Mastumoto et al. showed that the combination of arginine and Bifidobacterium spp. promote production of bacterial putrescine[29]. Kitada et al. further described a unique mechanism for polyamine production that requires the collaboration of several taxa group of the gut bacteria as shown in FIG. 4. [19]. The several taxa group of the gut bacteria are described below. First, the acid tolerant bacteria such as E. coli, possess an arginine dependent acid resistance system (arginine-agmatine antiporter) that exports intracellular agmatine and imports extracellular arginine and is strongly activated at pH below 6.5. The second type of gut bacterial is the bacteria comprising the energy production system such as Enterococcus faecalis (agmatine deiminase). These bacteria lack ODC or ADC but can import extracellular agmatine via the agmatine-putrescine antiporter to complete the polyamine and ATP production. Third, the acid producing bacteria (e.g., Bifidobacterium animalis subsp. lactis) maintain an environmental acidification. Agmatine is the key molecule in the cross feeding among these multiple bacteria that follow independent survival strategies [19]. Therefore, supplementation of arginine, optimization of ADC activity and acid production may be key factors for boosting agmatine and the polyamine production. FIG. 4 illustrates the hybrid mechanism for putrescine production pathway consisting of a cooperation between bacteria with acid-resistance system and bacteria with ATP synthesis system.

### Absorption and Distribution of Exogeneous Vs Microbiome-Derived Polyamines and Agmatine in the Gut

Polyamines in the gastrointestinal lumen have different origins such as from diet, intestinal microbiota, pancreatic-biliary secretions, and intestinal death cells. However, the precise contribution of each source to the whole polyamine pool is not known[18, 40].

The dietary polyamines (putrescine, spermidine and spermine) are rapidly absorbed in the lumen predominantly in the duodenum and jejunum[18]. A study focused on luminal polyamines concentration in normally fed male Sprague Dawley mice showed that putrescine concentration was the highest in the duodenum and upper jejunum (2000-3000 nmol/g wet tissue). The lowest concentration was in the ileum. The spermine and spermine concentrations were in average 5 time lower than the putrescine concentration. The maximum spermidine level was found in the cecum (˜700 nmol/g wet tissues) and was absent in the ileum. Spermine was in lowest concentration, only detectable in the duodenum and jejunum (100 to 200 nmol/g wet tissues)[17]. To further understand how polyamines are absorbed and redistributed in the billiarohepatic circulation, [¹⁴C] Putrescine (1.6 nmol) was injected in the intestinal lumen at various location and the radioactivity was followed in the pancreaticobiliary secretions. The highest radioactivity recovery was found after injection in lower and upper Jejunum within 60 minutes. Colonic injection induced an increase in radioactivity in the pancreaticobiliary secretions with the same time frame although the recovery was much lower than jejunum injection. Ileum injection profile was different as the radioactivity recovery was lower and longer with a plateau reached at around 140 minutes after injection suggesting that radioactivity is not absorbed in the ileum but reach the caecum and colon before being absorbed[17].

In human, putrescine, spermidine and spermine jejunal flow rate at fasting was around 7000, 2000 and 500 nmol respectively for a 20 min sampling period highlighting an important endogenous intestinal polyamine concentration. After a test meal, putrescine flow rate increased by 25% in jejunum but no significant changes was found for the other polyamines and no change was observed in ileum[18]. Up to 20% of putrescine in test meal is recovered in blood suggesting that a large portion was metabolized in the intestinal wall and/or in the liver[18]. Instead, acetylated putrescine and mild increase in spermine/spermidine was found [28].

The distribution of radioactivity after ingestion of [¹⁴C]-agmatine in rats showed that stomach is the main sight of exogenous agmatine absorption[15, 20]. The highest level of radioactivity was found in the liver and a large amount was also present in the stomach wall [20]. After 3-hour digestion period, radioactivity was detected in the lumen of the ileum and colon[20]. As it is unlikely that agmatine was transported to these gut segments without absorption, the authors suggested the presence of radioactivity may be associated to pancreatobiliary fluid circulation [15, 20]. The radioactivity level in blood was low and varied greatly among subjects highlighting a rapid redistribution of agmatine among the organs[20].

Mucosal and luminal polyamine synthesis attributed to mammalian and prokaryotic activities respectively were evaluated by monitoring the activity of ADC and ODC decarboxylases [17]. In general, luminal ADC activity was higher than ODC activity which confirm that bacterial polyamine production occurs mainly via ADC activity. Both ADC and ODC activity were almost not detectable in the duodenum and jejunum while ADC activity was the highest in stomach, caecum and colon, suggesting differential production of bacterial polyamines in the gut. Interestingly when luminal polyamine concentration was high, mucosal ODC rate was low, except for jejunum which presented high putrescine level and ODC activity[17].

### Distribution of Agmatine and Polyamines in Mammalian Tissues.

Agmatine, spermine and spermidine level in rats' tissues and human biofluids are summarized from different publications in Table 3. The concentration units have been standardized to μM for comparability purposes among papers. Agmatine is the most abundant in stomach, small intestine and large intestine tissues in Sprague-Dawley rats with a maximum level observed in the stomach at 0.071 μg/g wet tissue[22]. The same order of magnitude is observed in Male Long Evans Rats at Different Ages[22]. The spermidine and spermine concentrations in Wistar rats tissues are 10 to 100 times more abundant with the highest level found in small intestine (213 and 100 μg/g wet tissue respectively) and liver (230 and 235 μg/g wet tissue respectively)[23]. We cannot exclude that the variability in spermidine, spermine and agmatine concentrations may be due at least in part to the difference in rats species and the analytical methods used in the different publications [26, 43].

TABLE 3
Agmatine, spermine and spermidine level in rats tissues and human
biofluids summarized from different publications [22-26, 43, 44]

| | Rat | | | | | | | Human | | |
|---|---|---|---|---|---|---|---|---|---|---|
| | | Small | Large | Brain | Kidney | Liver | | | Feces | |
| | Stomach | Intestine | intestine | μg/g | μg/g | μg/g | | | μmol/g | |
| | μg/g wet | μg/g wet | μg/g wet | wet | wet | wet | Blood | Urine | dry | Serum |
| | tiss | tiss | tiss | tiss | tiss | tiss | (μM) | (μM) | matter | (μM) |
| Agmatine | 0.071 | 0.055 | 0.027 | 0.0024 | 0.0064 | 0.0056 | 0.0034 | 0.405 | 6 to 13 | 0.425 |
| Spermidine | 134 | 213 | 184 | 98 | — | 230 | 34.4-137 | 0.34-1.3 | 1.5-2.5 | 0.07-0.2 |
| Spermine | 77 | 100 | 130 | 98 | — | 235 | — | 0.148-5.53 | — | 0.09-0.15 |
| | | | | | | | | | | (blood) |

The rat blood concentration of agmatine, spermine and spermidine is significantly lower than the other tissues supporting the earlier observation that these molecules are rapidly redistributed and/or metabolized in the different tissues[22]. The pharmacokinetics, pharmacodynamic, (PK/PD) experiments showed agmatine half-life in rat blood after a bolus injection (50 mg/kg) is about 5 minutes[27]. Ten minutes after a radiolabeled polyamines intravenous (IV) injection in rat, plasma putrescine decreases by 67%, spermine by 30% and spermidine by 89%[26]. The systemic polyamines level may not be the best representation of the effective dose required for health benefits.

In human urine and serum, agmatine concentration is in the same range of magnitude as spermidine and spermine. Swanson et al. found that agmatine concentration exceeded spermidine concentration in human fecal samples with a range of 6 to 13 μmol/g dry matter[24, 25]. However, no further publication directly comparing the different polyamines in human biofluids and tissues are available to support these observations. The investigation of Nestlé and public semi-quantitative metabolomic data highlighted a large inter-individual variability in fecal agmatine level in infant and adults with IBD. Due to the lack of large and quantitative data in healthy adult population, it is difficult to draw conclusion on the distribution of polyamines and agmatine in human biofluids.

Comparison of Gut-Derived Polyamines and In-Vitro Polyamines Concentration from Batch Fermentation with Polyamine Oral Dose, Toxic Dose and Effective Dose.

Applicant has attempted to compare the gut-derived polyamine production with the average effective dose for a benefit and toxic dose by calculating a rough estimate of the daily human fecal and ileal content of polyamines and agmatine. Applicant has found that the gut-derived polyamines content is lower than the effective dose or toxic dose. However, the amounts of agmatine and spermidine in feces are higher than the concentrations required fora mechanistic effect. The comparison of gut-derived polyamines and in-vitro polyamines concentrations from batch fermentation with polyamine oral dose, toxic dose and effective dose are shown in Table 4.

TABLE 4
Comparison of gut-derived polyamines and in-vitro polyamines concentrations from
batch fermentation with polyamine oral dose, toxic dose and effective dose.

| | Putrescine | Spermidine | Agmatine |
|---|---|---|---|
| Feces (μmol/g | ~1.5 | ~2.0 | ~10 |
| dry matter) based | | | |
| on [24] | | | |
| Estimated daily | 4.9 | 8.1 | 30 |
| fecal content (mg) | | | |
| based on [24] | | | |
| Estimated fecal | 500 | 666 | 3333*** |
| concentration in | | | |
| wet feces | | | |
| (75% water) (μM) | | | |
| Estimated concentration | 87.9397 | 25.1256 | — |
| Jejunum (μM) based | | | |
| on [18] Flow rate: | | | |
| 79.6 ml/20 min | | | |
| Estimated concentration | 37.897 | 5.754 | — |
| in ileum (μM) based | | | |
| on [18] | | | |
| Flow rate: 50.4 ml/20 min | | | |
| Affinity for α-2 | — | — | 0.8-164 |
| adrenoreceptors | | | (depending on |
| (Ki) (μM) | | | organs/cell type) |
| Affinity for imidazoline | — | — | 0.33 to > 300 |
| receptors (Ki) (μM) | | | (depending on |
| | | | organs/cell type) |
| Cytoprotective effect | — | 10-100 | — |
| (μM) | | (depending on | |
| | | organs/cell type) | |
| daily dose based on | — | 1-4 mg | 0.5 to 1 g |
| synthetic form | | | |
| no observed effect level | 180 mg/kg body | 83 mg/kg body | 2.67 g daily |
| (max dose without | weight/day | weight/day | |
| toxicity)[33] | (~12 g) | (~5.4 g) | |
| Daily diet dose in UE | 211.9 | 87 | — |
| (μmol/day)[35] | | | |
| Average* concentration in | 6749.8 | 4.38 | 0.97 |
| Prodigest exp (μM) | | | |
| Average** concentration | 5141.7 | 11.95 | 35.29 |
| in house batch (μM) | | | |
| Average** concentration | 2399.2 | 1.19 | 3488.4 |
| in tube experiments (μM) | | | |

*Average calculated from concentration at 6 h + 24 h + 48 h − 0 h;
**average from concentration at 6 h + 24 h − 0 h; and
***No other reference in literature

### Health Benefits of Agmatine

The physiological relevance of endogenous agmatine is still unclear, but several health benefits have been attributed to the supplementation of synthetic agmatine. These benefits are ranked based on the level of evidences [6], including: 1) anti-depressive effects & improved cognition (multiple pre-clinical evidences on several animal models); 2) pain release and neuroprotection (Several pre-clinical evidences and a clinical trial); 3) vasodilatation and cardioprotective (several ex-vivo studies and few pre-clinical evidences); 4) improved metabolic health (stimulate fatty acid oxidation, glucose regulation) (few in-vitro studies and pre-clinical evidences); and 5) cellular health (reduction of oxidative stress, cytoprotection pro and antiproliferative effect) (few in-vitro studies).

More details on the level of evidences linked to the potential clinical applications of agmatine can be found in the review of Piletz et al [6].

### Mechanisms of Actions of Agmatine in Different Tissues.

The mechanisms related to agmatine benefits include anti-inflammatory, anti-apoptotic, anti-oxidant, inhibition of gliosis and edema, angiogenic, neurogenic and scavenging effects depending on the targeted tissue[6, 7]. These mechanisms have been linked to receptors and non-receptors-based effects of agmatine.

### Receptors Based Effects

Agmatine binds a range of receptors and biding sites with relatively high affinity, as listed in Table 5 below.

TABLE 5
Affinity or potency of agmatine at various receptors and binding sites[5].
Affinity or potency of agmatine at various receptors and binding sites

| | | |
|---|---|---|
| α₂-Adrenoceptors | Rat cerebral cortex | Kᵢ 4 μM |
| α₂-Adrenoceptors | Rat cerebral cortex | Kᵢ 7.9 ± 0.09 μM |
| α₂-Adrenoceptors | Rat cerebral cortex | Kᵢ 12.1 ± 2.5 μM |
| α₂-Adrenoceptors | Bovine adrenal cortex | Kᵢ 11-20 μM |
| α₂-Adrenoceptors | Bovine adrenal cortex | Kᵢ 17.0 ± 7.5 μM |
| α_{2A}-Adrenoceptors | Human recombinant receptor | Kᵢ 47 ± 2.6 μM |
| α_{2B}-Adrenoceptors | Human recombinant receptor | Kᵢ 164.4 ± 21.4 μM |
| α_{2C}-Adrenoceptors | Human recombinant receptor | Kᵢ 26.3 ± 4.0 μM |
| I₁-Imidazoline binding sites | Bovine ventrolateral medulla | Kᵢ 0.7 μM |
| I₁-Imidazoline binding sites | Bovine adrenomedullary cell | Kᵢ 0.127 ± 0.033 μM |
| I₁-Imidazoline binding sites | Human platelet-purified plasma membranes | Kᵢ 0.033 ± 0.019 μM |
| I₁-Imidazoline binding sites | Rat stomach membranes | Kᵢ 0.098 μM |
| I₂-Imidazoline binding sites | Rat aorta smooth muscle cells | Kᵢ 0.24 ± 0.025 μM |
| I₂-Imidazoline binding sites | Rat whole brain membranes | Kᵢ 417 ± 119 μM |
| I₂-Imidazoline binding sites | Rat stomach membranes | Kᵢ 266 μM |
| I₂-Imidazoline binding sites | Bovine adrenal chromaffin cells | Kᵢ 6.00 μM |
| I₂-Imidazoline binding sites | Bovine endothelial cells | Kᵢ 0.24 ± 0.025 μM |
| I₂-Imidazoline binding sites | Human platelet-purified internal membranes | Kᵢ 74.40 ± 14.2 μM |
| I₂-Imidazoline binding sites | Human atrial appendages | Kᵢ >100 μM |
| I₂-Imidazoline binding sites | Chicken brain membranes | Kᵢ 0.63 μM |
| I₂-Imidazoline binding sites | Frog whole brain membranes | Kᵢ 7.5 ± 2.6 μM |
| I₂-Imidazoline binding sites | Rabbit whole brain membranes | Kᵢ 856 ± 4 μM |
| Presynaptic imidazoline receptor | Human atrial appendages | Kᵢ >300 μM |
| Presynaptic imidazoline receptor | Rabbit aorta | Inhibition at 0.1 and 1 μM |
| NMDA receptor, polyamine binding site | Rat cerebral cortex | Kᵢ 14.8 μM |
| NMDA receptor, MK-801 binding site | Rat cerebral cortex | Kᵢ 219 μM |
| NMDA receptor | Rat cerebral membrane | Kᵢ 50 μM |
| NMDA receptor | Rat hippocampal neurons | Kᵢ −200 μM |
| NMDA receptor, NR1-ζ/NR2-ε1 | Recombinant receptor | IC₅₀ 286 ± 45 μM |
| NMDA receptor, NR1-ζ/NR2-ε2 | Recombinant receptor | IC₅₀ 330 ± 146 μM |
| NMDA receptor, NR1-ζ/NR2-ε3 | Recombinant receptor | IC₅₀ 326 ± 35 μM |
| NMDA receptor, NR1-ζ/NR2-ε4 | Recombinant receptor | IC₅₀ 289 ± 73 μM |
| 5-HT₃ receptor | Mouse N1E-115 cells | IC₅₀ 708 μM |
| 5-HT₃ receptor | Mouse N1E-115 cells | Kᵢ >1000 μM |
| σ2-Binding sites | Rat stomach membranes | Kᵢ 668 μM |
| σ2-Binding sites | Mouse N1E-115 cells | IC₅₀ >1000 μM |
| Nicotinic acetylcholine receptors | Chick retina | IC₅₀ >1000 μM |

Agmatine is an agonist of two types of GPCR receptors. The α-2 adrenoreceptors (also known as α-2 adrenoceptors or α-2 adrenergic receptor) (affinity: Ki 0.8-164 μM) and the Imidazoline receptors (affinity: Ki 0.33->300 μM) which trigger a range of signalling cascade. Four distinct sub-types of α-2 adrenoreceptors have been characterized (α-2A, α-2B, α-2C, α-2D) and found in various organs including brains, liver, gallbladder and gastrointestinal tract[45]. These receptors were identified in gut cell lines (HT29, Caco2-3B, enterochromaphin cells) and immune cells (Rat kupffer cells coll and peritoneal monocytes macrophages)[45-50]. The α-2 adrenoreceptors were primarily identified in pre- and post-synaptic neurons where they mediate the inhibition of the central and peripheral nervous systems. Imidazoline (IL1) receptors activation is linked with neuroprotective effects, increase in sodium and calcium excretion, urine flow rate and changes in gastric motility [9, 27, 51, 52]. It is also involved in regulation of blood pressure and may act synergistically with α-2 adrenoreceptors [51]. IL1 receptors are present in several organs including brains, liver, proximal digestive tract, GI tract (isolated piglet ileum, human colonic epithelial T 84 cell line) and in lymphoid tissues (ex: Human peripheral blood mononuclear cells) [53, 54].

These receptors regulate the phosphorylation of AKT& ERK1/2, CAMP & PKA and JNK1/2, triggering the subsequent activation of the transcription factors including CREB and NRF2[9, 55]. CREB binds to the DNA sequence cAMP response elements (CRE) and activates the transcription of genes regulating diverse cellular responses. It promotes the transcription of the brain-derived-neurotrophic factor (BDNF), a key player in synaptic plasticity and memory process[56]. CREB is found to limit pro-inflammatory responses in T and B cells by inhibiting NFkβ complex and activating IL-2, IL-6, IL-10, and TNF-a[11]. During fasting, CREB activates gluconeogenesis and fatty oxidation while suppressing lipid storage and synthesis in liver[57]. NRF2 is another transcription factor that activates a range of cytoprotective genes involved in oxidative stress response (GSH production, ROS detoxification) and anti-inflammation[10, 58].

Agmatine is an antagonist of N-methyl-D-aspartate (NMDA) receptors at high affinity (Table 5). The NMDA have an important role in synaptic plasticity and intracellular Ca2+ regulation[59]. Its activation can promote neuronal survival and resistance to trauma in healthy conditions but also triggers neuronal atrophy and cell death in pathological conditions (e.g. ischemia)[60]. NMDA antagonist have been associated with reduction of stress, and antidepressant effect in patients with treatment resistant effect[59, 60].

Importantly, there is no study suggesting the activation of imidazoline or α-2-adrenergic receptors by spermidine, spermine or putrescine. Arginine activates these receptors at much lower affinity (Ki 5 mM) suggesting that arginine effect on these receptors is probably mediated by agmatine[12]. However, spermidine and spermine can also inhibit the NMDA receptors[61].

### Non Receptors-Based Effect

In several ex-vivo and in-vitro cell assays, agmatine interferes with polyamine metabolism, nitric oxide synthase and promotes antiproliferative effects at millimolar level (1 mM for mouse kidney proximal tubule, Ras transformed NIH-3T3 fibroblast, mouse glomerular mesangial, human Schwann tumor, rat glomerular endothelial cells, human colon cancer HT29 cell and 0.01 mM for HTC rat hepatoma cells)[41]. The intracellular concentration of agmatine is concomitant with a decrease in rate-limiting polyamine biosynthetic enzyme ODC activity, as well as a decrease in polyamine uptake and intra-cellular level of putrescine and spermidine. This may be linked to agmatine-induced activation of the protein antizyme which is involved in the auto-regulation of the polyamines contents in cells[62]. By contrast agmatine is thought to activate polyamine catabolism via the activation of spermidine/spermine acetyltransferase (SSAT) resulting in the increase in acetylated polyamines[28]. The acetylation of polyamines reduces their charge, altering the ability to bind with other macromolecules and modify their functions. Acetylated polyamines can be further oxidized by acetylpolyamine oxidase or readily excreted from the cells[63]. An injection of 456.6 mg/kg of agmatine (i.p) in suiss female mice confirms the inhibition of ODC and activation of SSAT in liver and kidney and a partial reduction of epithelial cell proliferation in kidney renal tubule[64]. Based on these observations, agmatine is considered as a potential tumor suppressor, however further pre-clinical studies are required to confirm the anti-proliferative effect of agmatine at low and high dose.

### Host Benefits of Boosting Microbiome Derived Polyamines

Mastumoto and colleagues studied the role of microbial polyamines on health benefits in several preclinical and clinical experiments[19, 25, 29-31]. They found that arginine intake (from 0 to 9 mg/g body weight) increased the fecal concentration of putrescine and spermidine which was abolished after antibiotics use[31]. Long term intervention (6 months) with oral administration of arginine, probiotic bifidobacteria LKM512 or both was also tested. The double treatment reduced DNA damage (urinary 8-OHDG), improved senescence marker (SMP-30), and longevity and decrease age-related memory loss by comparison to the control and the single treatment [31].

In a randomized placebo-controlled trial, the same authors showed that 12 weeks intervention with a yoghourt containing arginine (600 mg) and Bifidobacterium animalis subsp. Lactis improved endothelial function (increased reactive hyperemia index) in healthy individuals with BMI of 25 (maximum value in the healthy range)[30]. In addition, the concentrations of fecal putrescine and serum spermidine were significantly higher in the treatment group than those in the placebo group[30].

In a mouse model of osteoporosis, Chevalier et al. demonstrated that warm exposure improved bone health by increasing trabecular bone volume, connectivity density, and thickness[32]. The microbial transplantation of warm treated mice to non-treated mice reproduced these warm-induced bone effects. Further metabolomics and metagenomics analysis showed warm-associated changes in the microbiome composition (expansion of Akkermansia muciniphila and genera from Bacteroides and Alistipes and decline of bacteria from the Muribaculaceae or Lachnospirae genera) and an increase in bacterial polyamine synthesis capability resulting in higher cecal and fecal polyamines content. Polyamines synthesis inhibition with diminazene aceturate limited the warm-induced beneficial effects[32]. Boosting the microbiome-derived polyamine biosynthetic pathways may have a systemic effect on the host. However, the relationship between the intestinal polyamines and the targeted benefits (memory loss, bone structure, endothelial function) remain unclear as no evidence on the intestinal polyamine transport to the sight of action have been shown.

The present disclosure provides probiotics, compositions and methods for increasing the production of agmatine using microbiota in order to provide certain health benefits to a subject.

### EXAMPLES

The following non-limiting examples support the concept of using the microbiota to increase the production of agmatine.

### Example 1

### Material and Methods

### Upper GIT Digestion of Selected Products

One test product (Protein 1) was subjected to a full passage through the oral, gastric and small intestinal phase, the latter involving absorption. This was considered important as this product contains a fraction of digestible compounds that, in vivo, is absorbed at the level of the small intestine following the conversion to small molecules. To ensure the quality of its digestion protocols, ProDigest updated its digestion methods based on a consensus protocol, developed within a large European framework (COST Action InfoGest). The latter describes a static digestion method with the aim to enhance comparison of digestion experiments across research teams (Mackie and Rigby, 2015) 3. ProDigest further improved this digestion method by incorporating more accurate pH profiles together with a simulation of the small intestinal absorption by means of a dialysis approach. This simulation of small intestinal absorption via dialysis enables the removal of small molecules from intestinal digests. To do so, a 14 kDa dialysis membrane was used.

### Preservation of Fecal Samples

Fecal material was collected from five healthy adult donors. Fecal suspensions were prepared and mixed with an internally optimized cryoprotectant. The obtained suspensions were aliquoted, flash frozen and then preserved at −80° C. (cryostock). Just before the experiment, fecal samples were defrosted and immediately added to the reactors.

Preparation of the cryostock from a single fecal suspension ensures that identical microbial communities are obtained in each aliquot, and thus that an identical inoculum is used throughout the different project phases. Moreover, preservation of aliquots ensures that the preserved samples undergo only one freeze-thawing cycle before introduction in a given incubation, as a new aliquot is used for each phase of the project. These actions ensure optimal reproducibility.

### Short-Term Colonic Incubations

A short-term screening assay typically consists of a colonic incubation of a single dose of a test compound under conditions representative for the proximal large intestine, using bacterial inocula from selected donors as microbial sources.

At the start of the short-term colonic incubations, test ingredients were added to a sugar-depleted nutritional medium containing basal nutrients of the colon. Because nutrients of this sugar-depleted nutritional medium will also be fermented by the colonic microbiota, a blank containing only the sugar-depleted nutritional medium (without product) was included for each donor. Finally, fecal inocula of five donors were added (healthy adult humans with no history of antibiotic use 6 months preceding the experiment). Five treatments and one blank were tested per donor, resulting in the experimental set-up provided in Table 6.

TABLE 6
Experimental sample set up.

| Donor 1-5 | Condition | Arginine | FOS | Protein 1 |
|---|---|---|---|---|
| 1 | Blank | / | / | / |
| 2 | Arginine | 3 g/L | / | / |
| 3 | FOS | / | 3 g/L | / |
| 4 | Arginine + FOS | 3 g/L | 3 g/L | / |
| 5 | Protein 1 | / | / | ≤6 g/L |
| 6 | Protein 1 + FOS | / | 3 g/L | ≤6 g/L |

Incubations were performed in single repetition, resulting in 30 independent incubations. Reactors were incubated for 48 h at 37° C., under shaking (90 rpm) and anaerobic conditions. The incubations were performed in fully independent reactors with sufficiently high volume in order to not only ensure robust microbial fermentation, but also to allow the collection of multiple samples over time. Sample collection enables assessment of metabolite production and thus to understand the complex microbial interactions that are taking place.

### Endpoints of the Study

An assessment was made of the change in pH, and of gas, SCFA, ammonium and lactate production at the start of the incubation and after 6 h, 24 h and 48 h. Quantitative deep shotgun sequencing was performed at the start and after 24 h and 48 h of incubation. Targeted metabolic analysis of eight polyamines, an amino acid and a neurotransmitter was performed at the start of the incubation, and after 6 h, 24 h and 48 h.

### Determination of Protein and Peptide/AA Fractions in Protein 1

The Kjeldahl procedure and TCA precipitation were applied to determine the protein and peptide/AA fractions in original product Protein 1. Similarly, the small intestinal solution after upper GI passage was analyzed as such, and the supernatant after TCA precipitation, to determine both product fractions. These calculations allowed to deduce the factual product concentrations that were administered to the colonic incubations.

### Overall Fermentative Activity

pH: The degree of acidification during the experiment is a measure for the intensity of bacterial metabolism. The pH of the incubations provides a rough indication on the speed of fermentation of the different test products.

Gas production: The colon incubations were performed in closed incubation systems. This allows to evaluate the accumulation of gases in the headspace, which can be measured with a pressure meter. Gas production is a measure of microbial activity, and thus of the speed of fermentation of the potentially prebiotic substrates. H₂ and CO₂ are the first gasses to be produced upon microbial fermentation; they can subsequently be utilized as substrates for CH₄ production, reducing the gas volume. H₂ can also be utilized to reduce sulfate to H₂S, resulting from proteolytic fermentation 4. As a result, N₂, O₂, CO₂, H₂ and CH₄ constitute for 99% the volume of intestinal gas. The remaining 1% consists of NH₃, H₂S, volatile amino acids and short chain fatty acids.5

### Changes in Microbial Metabolites

Short chain fatty acid analysis: The pattern of SCFA production is an assessment of the microbial carbohydrate metabolism (acetate, propionate and butyrate) or protein metabolism (branched SCFA) and can be compared to typical fermentation patterns for normal GI microbiota.

Lactate analysis: The human intestine harbors both lactate-producing and lactate-utilizing bacteria. Lactate is produced by lactic acid bacteria and decreases the pH of the environment, thereby also acting as an antimicrobial agent. Protonated lactic acid can penetrate the microbial cell, after which it dissociates and releases protons within the cell, resulting in acidification and microbial cell death. It can also be rapidly converted into propionate and butyrate by other microorganisms.

Ammonium analysis: Ammonium is a product of proteolytic degradation. Proteolytic fermentation results in the production of potentially toxic or carcinogenic compounds such as p-cresol and p-phenol. Ammonium can be used as an indirect marker for low substrate availability.

Targeted metabolic analysis: Eight different polyamines were targeted, more specifically putrescine, agmatine, acetyl-agmatine, omithine, spermidine, spermine, citrulline and 4-guanidinobutanoic acid. Additionally, samples were analyzed for amino acid arginine and neurotransmitter gamma-Aminobutyric acid (GABA).

### Microbial Community Composition and Functional Analysis

Initial QC, adapter trimming and preprocessing of metagenomic sequencing reads were done using BBduk. The quality-controlled reads were then subjected to a translated search using Diamond against a comprehensive and non-redundant protein sequence database, UniRef 90. The UniRef90 database, provided by UniProt, represents a clustering of all non-redundant protein sequences in UniProt, such that each sequence in a cluster aligns with 90% identity and 80% coverage of the longest sequence in the cluster. The mapping of metagenomic reads to gene sequences are weighted by mapping quality, coverage and gene sequence length to estimate community wide weighted gene family abundances as described by Franzosa et al6. Gene families were then annotated to MetaCyc reactions (Metabolic Enzymes) to reconstruct and quantify MetaCyc metabolic pathways in the community as described by Franzosa et al. Furthermore, the UniRef_90 gene families are also regrouped to GO terms in order to get an overview of GO functions in the community. Lastly, to facilitate comparisons across multiple samples with different sequencing depths, the abundance values are normalized using Total-sum scaling (TSS) normalization to produce “Copies per million” (analogous to TPMs in RNA-Seq) units.

### Quantification of Total Bacterial Cells by Flow Cytometry

Samples that were analyzed with shotgun sequencing were also analyzed with flow cytometry to determine the number of total bacterial cells, thus allowing to convert the proportional values obtained with shotgun sequencing into absolute quantities by multiplying relative abundances of any population (at any phylogenetic level) in a sample with the total cell count obtained with FC of the given sample. Samples were analyzed on a BD Facs verse. The samples were run using the high flow rate. Bacterial cells were separated from medium debris and signal noise by applying a threshold level of 200 on the SYTO channel. Proper parent and daughter gates were set to determine all populations.

### Statistics

To assess whether differences between treatment effects in terms of the investigated endpoints were statistically relevant, paired two-sided T-tests were performed, taking into account all donors. To control the proportion of false discoveries when conducting a high number of comparisons, the Benjamini-Hochberg false discovery rate (FDR) was applied. Differences between treatment effects were considered significant when the obtained p-value (obtained through the paired two-sided T-test) was smaller than a reference value (ref). This reference value was obtained by ranking of obtained p-values in ascending order within the donor group. The rank of a given p-value was termed (i) and varied between 1 to the total amount of p-values (m=5; as mentioned below). The reference value was calculated by multiplying the FDR with the rank of the p-value, divided by the total amount of comparisons made (ref=FDR*i/m). To compare treatment effects in terms of changes in pH, gas pressure, microbial metabolite production (SCFA, lactate, ammonium and biogenic amines), functional profiles and microbial community composition the FDR was set at 0.1, meaning that the lowest p-value should be below 0.02 to be significantly different, the second lowest below 0.04, etc. All calculations were carried out via Microsoft Excel. The following five comparisons were made to assess:

Effects of arginine, Protein 1 and FOS: differences between these treatments and respective blanks were calculated (3 comparisons).

Effects of FOS combined with arginine or Protein 1: differences between combined treatments and respective individual treatments (arginine and Protein 1) were calculated (2 comparisons).

A systematic representation of these five comparisons is provided in Table 7.

TABLE 7
Systematic representation of the five comparisons that
were made between treatment and respective reference
conditions to evaluate treatment effects on metabolic
markers and microbial community composition.

| | | | | | Protein |
|---|---|---|---|---|---|
| | | | | Arg + FOS − | 1 + FOS − |
| | Arg | Protein 1 | FOS | Arg | Protein 1 |
| All | Arg- | Protein 1- | FOS- | Arg + FOS − | Protein 1 + |
| Donors | Blank | Blank | Blank | Arg | FOS − |
| | | | | | Protein 1 |

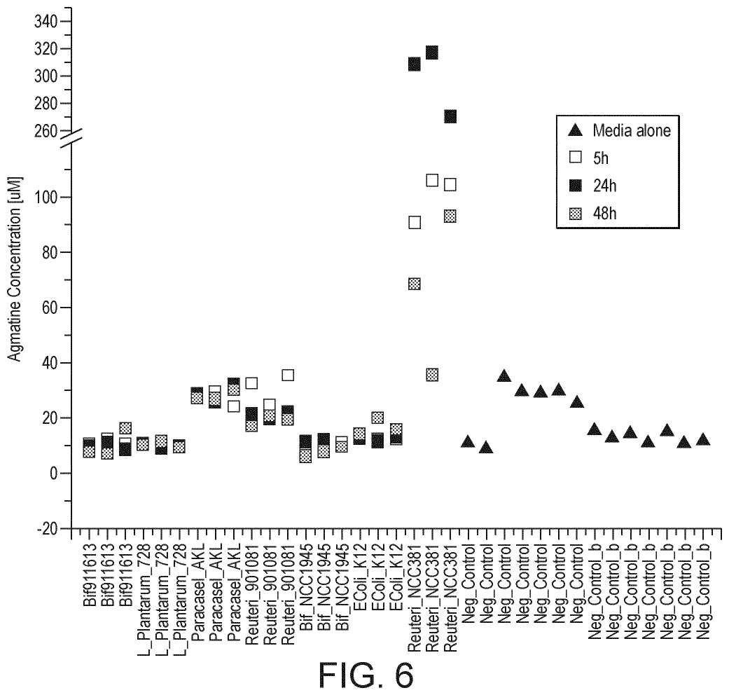

The experimental results are shown in FIG. 5. The test results in FIG. 5 clearly demonstrated that all the five donors produced agmatine in low concentrations when only arginine was added to the stool samples. Without FOS (acidifying compound), donors A and E produced about 5 μM of agmatine; donor B produced about 3 μM of agmatine; while donors C and D did not produce agmatine.

Addition of FOS in combination of arginine to the stool samples of the five donors significantly increased the production of agmatine up to about 10 times. The samples from donors A, B, C, D and E each produced about 12 μM, 5 μM, 17 μM, 8 μM and 20 μM of agmatine respectively, as shown in FIG. 5. The test results indicate that lower pH increased the production of agmatine by altering the background metabolism. After 24 hours, the abundance of agmatine was fully consumed.

### Example 2

### Analytical Test Results

### Objectives

To support potential product launch targeting agmatine production boost, analytical work was required in the present disclosure. The main objectives of the analytical work were to: 1) set-up a new analytical platform within the group; 2) develop an analytical approach for the quantification of agmatine and semi-quantification of additional polyamines in bacterial media; and 3) analyze and quantify the agreed compounds in samples (n=611) received from the HMI group 4—Provide analytical recommendations.

### Samples

Samples were generated in the host-microbe interaction group within the Institute of Health Sciences or externally. They were produced in various batches of experiments and collected in Eppendorf tubes. Samples were shipped in batches for analysis to the EPFL site and stored at −80° C. until day of analysis after each experimental trial performed.

### Analytical Approach

Access to a liquid chromatography hyphenated to a high-resolution mass spectrometer (LC-HRMS) was generously offered from the Proteins and peptides group within NIFSAS. Analytical work has been conducted to: 1) quantify agmatine and 2) only semi-quantify additional polyamine compounds and compounds related to the metabolic pathway (i.e. acetyl agmatine, γ-aminobutyric acid, citrulline, arginine, ornithine putrescine, spermidine and spermine) applying the external calibration approach.

### Analytical Methodology

### Sample Preparation

All samples were prepared according to the analytical approach developed in-house as reported in the R&D Memo: “Polyamines in bacterial media—Analytical approach using LC-HRMS”. Calibration ranges were defined as developed and then adjusted according to the calculated concentration of each analyte found in the unknown samples and the analytical limitations, to minimize >ULOQ results.

### Experimental Results

### Analytical Evaluation

Each analytical series was evaluated for each compound of interest. In each analytical series, a calibration curve with 6 QC samples at 3 different levels were injected with the unknown samples. When the number of unknown samples were >80, then a second calibration curve with additional 3 QC samples were injected at the end of the analytical sequence. R² of the calibration curve individual standard point CV as well as CVs of QC were checked according to the R&D Memo mentioned previously.

### Single Strain Selection and Time-Course Experiment (77 Samples Received and Analyzed)

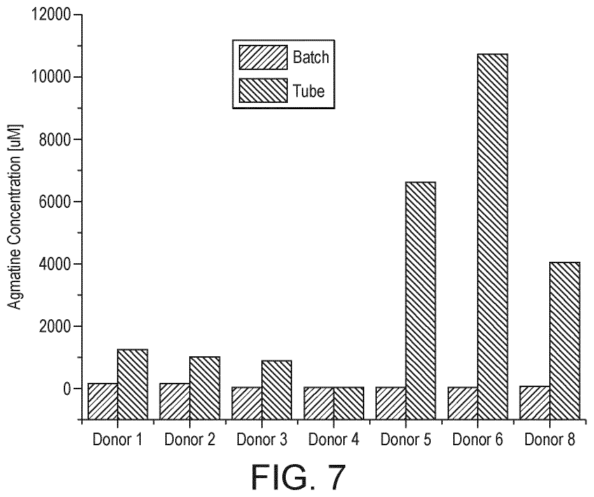

The objective of this experiment was to identify the most relevant time point for potential agmatine production. For this purpose, 6 different single strain were incubated in anaerobic conditions to evaluate their effect on agmatine production. Samples at 5 hours (T5 h), 24 hours (T24 h) and 48 hours (T48 h) were measured. The test results for evolution of agmatine concentration over time (T5 h, T24 h, T48 h) with strain screening are shown in FIG. 6. From FIG. 6, the agmatine concentrations reached a maximum at T24 h before decreasing. Further analysis was focused on T24 h for potential significant agmatine production.

### Example 3

### “Tube Vs Batch” Fermentation (48 Samples Received and Analyzed)

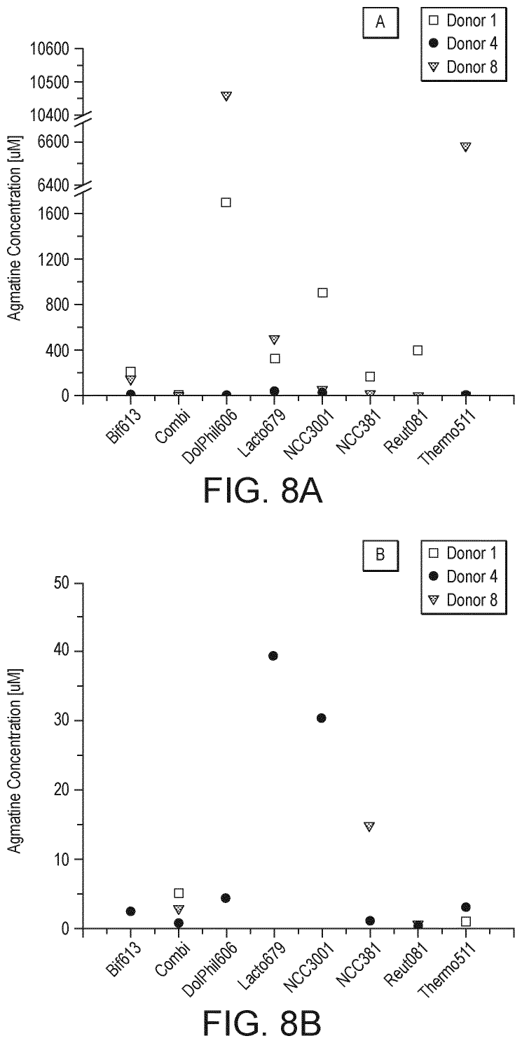

The objective of this experiment was to select the best conditions of in-house fermentation to obtain the highest agmatine concentration. For this purpose, 8 donors (faeces extracts) were selected and incubated to evaluate the best fermentation conditions. 7 donor extracts were analyzed at 0 hours (T0 h), 6 hours (T6 h) and 24 hours (T24 h). The test results for the tube and batch fermentation effects on the agmatine concentration are shown in FIG. 7. The test results in FIG. 7 clearly demonstrated that tubes fermentation exhibited the highest agmatine production (up to 10 times more than batch fermentation). This was the choice of fermentation for further experiments.

### Example 4

### Tube/Strain Combination (447 Samples Received; 200 Samples Analyzed)

The objective of this experiment was to investigate if combining specific strains with arginine could lead to an increase of agmatine production.

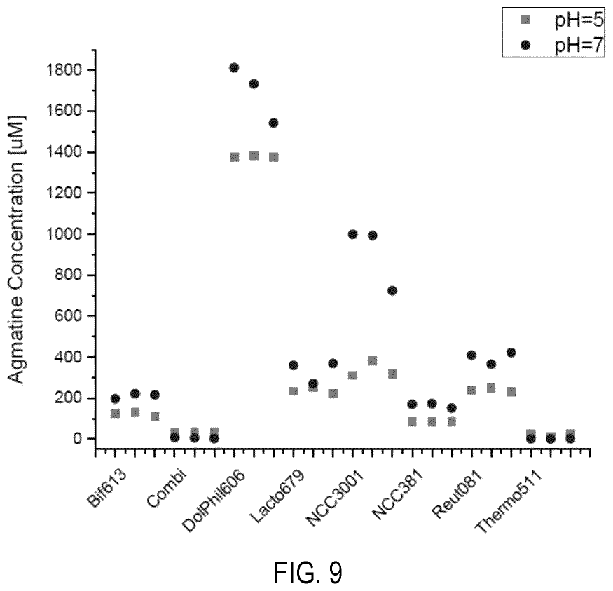

For this purpose, 3 donors from previous experiments were selected (low, mid and high agmatine producers) and incubated with 7 different single strains and 1 strain combination. Priority samples were identified within the team and samples at 0 hours (T0 h), 6 hours (T6 h) and 24 hours (T24 h) of specific donors (Donor 1, 4 and 8) were analyzed. The test results for effects of strain with various donors on agmatine production are shown in FIGS. 8A and 8B respectively to (A) represent the overall scale and (B) represent a zoom scale from 0 to 50 μM. Agmatine concentration increased in the 3 donors w while incubated with specific strains, as illustrated in FIGS. 8A and 8B.

The test results in FIGS. 8A and 8B clearly demonstrated that the following observation: 1) A positive effect on agmatine concentration was observed while Dolphilus 606, NCC3001 were incubated with Donor 1; 2) A positive effect on agmatine concentration was observed while Lacto679, NCC3001 were incubated with Donor 4; and 3) A positive effect on agmatine concentration was observed while Dolphilus 606, Thermo511 were incubated with Donor 8. This variability may suggest a donor dependent response after arginine supplementation on the agmatine production.

Remaining samples were kept stored at −80° C. for analysis in case the team is requiring them. These results allowed designing 4 various mixes to be incubated with specific donors for the selection of a blend mix to be produced.

### Example 5

### Impact of pH

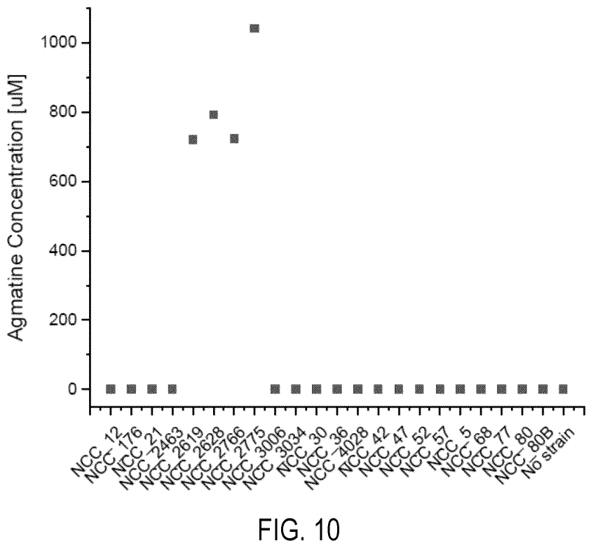

In the tube/strain combination experiments, two pH conditions at 5.0 and 7.0 (pH5 and pH7) were evaluated. FIG. 9 displays the results for impact of different pH conditions on agmatine concentration obtained for Donor 1. The test results in FIG. 9 clearly demonstrated that pH7 conditions always led to higher agmatine production (up to 3 times compared to pH5 conditions) except when applying strain Thermo511 of combination of strains. The pH control whenever possible was key in the agmatine production. In the presence of a bacteria, the neutral pH conditions at 7.0 led to higher agmatine production in the samples.

### Example 6

### Strain Investigation (24+36 Samples)

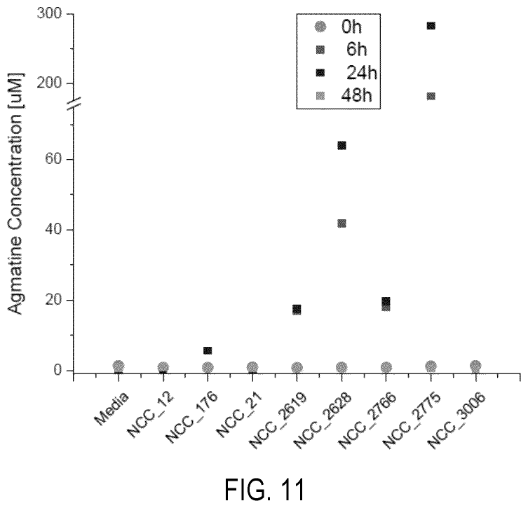

The objective of these experiments was to evaluate to different NCC strains and obtain potential candidate as agmatine production booster. For this purpose, donor 1 from previous experiment was selected (mid agmatine producer) and incubated with 22 different NCC strains in a first batch. T24 h samples were analyzed. The test results on effect of various strains incubation with donor 1 on agmatine concentration are shown in FIG. 10 which clearly demonstrated that 4 specific strains (NCC2619, NCC2628, NCC2766 and NCC2775) were drastically boosting the agmatine concentration at T24 h as displayed in FIG. 10.

To confirm the beneficial effect on agmatine production, a second experiment studying the incubation of a reduced number of strains incubated with donor 1. T0, T6 h, T24 h and T48 h samples were analyzed and test data on effect of specific strains incubation with donor 1 on agmatine concentration are presented in FIG. 11.

The test results in FIG. 11 clearly confirmed the previous findings that the 4 specific strains (NCC2619, NCC2628, NCC2766 and NCC2775) were drastically boosting the agmatine production with a maximum agmatine concentration at 24 hours (in these specific sampling conditions).

### Example 7

The objective of this experiment was to evaluate the effects of 4 different Lactobacillus acidophilus strains on the production and of agmatine and the bioavailability of agmatine using in vitro fermentation of arginine. In vitro fermentation of arginine was performed using stool samples from 5 donor candidates prepared in and tested in Example 1. The microbiota was supplemented with 4 different strains of Lactobacillus acidophilus (NCC2619, NCC2628, NCC2766 and NCC2775).

The concentrations of agmatine were assessed in a time course of 48 hours, to assess the bioavailability of agmatine.

All 4 strains of Lactobacillus acidophilus (NCC2619, NCC2628, NCC2766 and NCC2775) increased the overall concentrations and thus the bioavailability of agmatine. Such increased concentrations are associated to a decrease of the pH.

### CONCLUSION

The polyamines methodology using LC-HRMS developed and reported above was fit-for purpose for measuring 385 samples received from stream 1 within the framework of project Magma. Absolute quantification of agmatine allowed Applicant to select the best fermentation conditions and strains to increase the agmatine concentration. All the polyamines and related compounds (such as acetyl agmatine, γ-aminobutyric acid, citrulline, arginine, agmatine, omithine, putrescine, spermidine and spermine) were also measured in each and every sample.

Various changes and modifications to the presently preferred embodiments disclosed herein will be apparent to those skilled in the art. Such changes and modifications can be made without departing from the spirit and scope of the present subject matter and without diminishing its intended advantages. It is therefore intended that such changes and modifications be covered by the appended claims.

### REFERENCES

1. Madeo, F., et al., Spermidine: a physiological autophagy inducer acting as an anti-aging vitamin in humans, Autophagy, 2019. 15(1): p. 165-168.
2. Igarashi, K. and K. Kashiwagi, The functional role of polyamines in eukaryotic cells. Int J Biochem Cell Biol, 2019. 107: p. 104-115.
3. Thomas, T. and T. J. Thomas, Polyamine metabolism and cancer. J Cell Mol Med, 2003. 7(2): p. 113-26.
4. Madeo, F., et al., Spermidine in health and disease. Science, 2018. 359(6374).
5. Molderings, G. J. and B. Haenisch, Agmatine (decarboxylated L-arginine): physiological role and therapeutic potential. Pharmacol Ther, 2012. 133(3): p. 351-65.
6. Piletz, J. E., et al., Agmatine: clinical applications after 100 years in translation. Drug Discov Today, 2013. 18(17-18): p. 880-93.
7. Freitas, A. E., V. B. Neis, and A. L. S. Rodrigues, Agmatine, a potential novel therapeutic strategy for depression. Eur Neuropsychopharmacol, 2016. 26(12): p. 1885-1899.
8. Laube, G. and H. G. Bernstein, Agmatine: multifunctional arginine metabolite and magic bullet in clinical neuroscience, Biochem J, 2017. 474(15): p. 2619-2640.
9. Raasch, W., et al., Biological significance of agmatine, an endogenous ligand at imidazoline binding sites. Br J Pharmacol, 2001. 133(6): p. 755-80.
10. Tonelli, C., I. I. C. Chio, and D. A. Tuveson, Transcriptional Regulation by Nrf2. Antioxid Redox Signal, 2018. 29(17): p. 1727-1745.
11. Wen, A. Y., K. M. Sakamoto, and L. S. Miller, The role of the transcription factor CREB in immune function. J Immunol, 2010. 185(11): p. 6413-9.
12. Joshi, M. S., et al., Receptor-mediated activation of nitric oxide synthesis by arginine in endothelial cells. Proc Natl Acad Sci USA, 2007. 104(24): p. 9982-7.
13. Bassard, J. E., et al., Phenolamides: bridging polyamines to the phenolic metabolism. Phytochemistry, 2010. 71(16): p. 1808-24.
14. Galgano, F., et al., Focused review: agmatine in fermented foods. Front Microbiol, 2012. 3: p. 199.
15. Molderings, G. J., et al., Gastrointestinal uptake of agmatine: distribution in tissues and organs and pathophysiologic relevance. Ann N Y Acad Sci, 2003. 1009: p. 44-51.
16. Milovic, V., Polyamines in the gut lumen: bioavailability and biodistribution. Eur J Gastroenterol Hepatol, 2001. 13(9): p. 1021-5.
17. Osborne, D. L. and E. R. Seidel, Gastrointestinal luminal polyamines: cellular accumulation and enterohepatic circulation. Am J Physiol, 1990. 258(4 Pt 1): p. G576-84.
18. Benamouzig, R., et al., Fasting and postprandial polyamine concentrations in the human digestive lumen. Am J Clin Nutr, 1997. 65(3): p. 766-70.
19. Kitada, Y., et al., Bioactive polyamine production by a novel hybrid system comprising multiple indigenous gut bacterial strategies. Sci Adv, 2018. 4(6): p. eaat0062.
20. Molderings, G. J., et al., Exposure of rat isolated stomach and rats in vivo to [(14) C]agmatine: accumulation in the stomach wall and distribution in various tissues. Fundam Clin Pharmacol, 2002. 16(3): p. 219-25.
21. Heinen, A., et al., Pharmacological characteristics of the specific transporter for the endogenous cell growth inhibitor agmatine in six tumor cell lines. Int J Colorectal Dis, 2003. 18(4): p. 314-9.
22. Raasch, W., et al., Agmatine, the bacterial amine, is widely distributed in mammalian tissues. Life Sci, 1995. 56(26): p. 2319-30.
23. Jaenne, J., A. Raina, and M. Siimes, Spermidine and Spermine in Rat Tissues at Different Ages. Acta Physiol Scand, 1964. 62: p. 352-8.
24. Swanson, K. S., et al., Fructooligosaccharides and Lactobacillus acidophilus modify bowel function and protein catabolites excreted by healthy humans. J Nutr, 2002. 132(10): p. 3042-50.
25. Matsumoto, M., K. Kakizoe, and Y. Benno, Comparison of fecal microbiota and polyamine concentration in adult patients with intractable atopic dermatitis and healthy adults. Microbiol Immunol, 2007. 51(1): p. 37-46.
26. Rosenthal, S. M. and C. W. Tabor, The pharmacology of spermine and spermidine; distribution and excretion. J Pharmacol Exp Ther, 1956. 116(2): p. 131-8.
27. Raasch, W., et al., Agmatine, an endogenous ligand at imidazoline binding sites, does not antagonize the clonidine-mediated blood pressure reaction. Br J Pharmacol, 2002. 135(3): p. 663-72.
28. Rosenblum, M. G. and D. H. Russell, Conjugation of radiolabeled polyamines in the rat. Cancer Res, 1977. 37(1): p. 47-51.
29. Matsumoto, M., et al., Longevity in mice is promoted by probiotic-induced suppression of colonic senescence dependent on upregulation of gut bacterial polyamine production. PLoS One, 2011. 6(8): p. e23652.
30. Matsumoto, M., Y. Kitada, and Y. Naito, Endothelial Function is improved by Inducing Microbial Polyamine Production in the Gut: A Randomized Placebo-Controlled Trial. Nutrients, 2019. 11(5).
31. Kibe, R., et al., Upregulation of colonic luminal polyamines produced by intestinal microbiota delays senescence in mice. Sci Rep, 2014. 4: p. 4548.
32. Chevalier, C., et al., Warmth Prevents Bone Loss Through the Gut Microbiota. Cell Metab, 2020. 32(4): p. 575-590 e7.
33. Til, H. P., et al., Acute and subacute toxicity of tyramine, spermidine, spermine, putrescine and cadaverine in rats. Food Chem Toxicol, 1997. 35(3-4): p. 337-48.
34. Haenisch, B., et al., Regulatory mechanisms underlying agmatine homeostasis in humans. Am J Physiol Gastrointest Liver Physiol, 2008. 295(5): p. G1104-10.
35. Bardócz, S., et al., Polyamines in food—implications for growth and health. Journal of Nutritional Biochemistry, 1993.
36. Kralj Cigic, I., et al., Accumulation of Agmatine, Spermidine, and Spermine in Sprouts and Microgreens of Alfalfa, Fenugreek, Lentil, and Daikon Radish. Foods, 2020. 9(5).
37. Wang, W., H. D. Snooks, and S. Sang, The Chemistry and Health Benefits of Dietary Phenolamides. J Agric Food Chem, 2020. 68(23): p. 6248-6267.
38. Lortie, M. J., et al., Agmatine, a bioactive metabolite of arginine. Production, degradation, and functional effects in the kidney of the rat. J Clin Invest, 1996. 97(2): p. 413-20.
39. Zhu, M. Y., et al., Expression of human arginine decarboxylase, the biosynthetic enzyme for agmatine. Biochim Biophys Acta, 2004. 1670(2): p. 156-64.
40. Ramos-Molina, B., et al., Dietary and Gut Microbiota Polyamines in Obesity- and Age-Related Diseases. Front Nutr, 2019. 6: p. 24.
41. Mayeur, C., et al., Effects of agmatine accumulation in human colon carcinoma cells on polyamine metabolism, DNA synthesis and the cell cycle. Biochim Biophys Acta, 2005. 1745(1): p. 111-23.
42. Burrell, M., et al., Evolution and multiplicity of arginine decarboxylases in polyamine biosynthesis and essential role in Bacillus subtilis biofilm formation. J Biol Chem, 2010. 285(50): p. 39224-38.
43. Zhao, S., et al., Determination of agmatine in biological samples by capillary electrophoresis with optical fiber light-emitting-diode-induced fluorescence detection. J Chromatogr A, 2006. 1123(1): p. 138-41.
44. Forget, P., et al., Fecal polyamine concentration in children with and without nutrient malabsorption. J Pediatr Gastroenterol Nutr, 1997. 24(3): p. 285-8.
45. Bouscarel, B., et al., alpha 2-Adrenoceptors in the HT 29 human colon adenocarcinoma cell line: characterization with [3H]clonidine; effects on cyclic AMP accumulation. Eur J Pharmacol, 1985. 107(2): p. 223-31.
46. Schaak, S., et al., Alpha(2) adrenoceptors regulate proliferation of human intestinal epithelial cells. Gut, 2000. 47(2): p. 242-50.
47. Zhou, M., et al., The role of Kupffer cell alpha(2)-adrenoceptors in norepinephrine-induced TNFalpha production. Biochim Biophys Acta, 2001. 1537(1): p. 49-57.
48. Sud, R., et al., Antinociception occurs with a reversal in alpha 2-adrenoceptor regulation of TNF production by peripheral monocytes/macrophages from pro- to anti-inflammatory. Eur J Pharmacol, 2008. 588(2-3): p. 217-31.
49. Bellono, N. W., et al., Enterochromaffin Cells Are Gut Chemosensors that Couple to Sensory Neural Pathways. Cell, 2017. 170(1): p. 185-198 e16.
50. Szelenyi, J., et al., Contribution of differently localized alpha 2- and beta-adrenoceptors in the modulation of TNF-alpha and IL-10 production in endotoxemic mice. Ann N Y Acad Sci, 2000. 917: p. 145-53.
51. Maltsev, A. V., et al., Alpha-2 adrenoceptors and imidazoline receptors in cardiomyocytes mediate counterbalancing effect of agmatine on NO synthesis and intracellular calcium handling. J Mol Cell Cardiol, 2014. 68: p. 66-74.
52. Zadori, Z. S., et al., Evidence for the gastric cytoprotective effect of centrally injected agmatine. Brain Res Bull, 2014. 108: p. 51-9.
53. Molderings, G. J., et al., Imidazoline recognition sites and stomach function. Ann N Y Acad Sci, 1999. 881: p. 332-43.
54. Li, X., et al., Agmatine Protects Against the Progression of Sepsis Through the Imidazoline 12 Receptor-Ribosomal S6 Kinase 2-Nuclear Factor-kappaB Signaling Pathway. Crit Care Med, 2020. 48(1): p. e40-e47.
55. Zhang, J., M. M. El-Mas, and A. A. Abdel-Rahman, Imidazoline I(1) receptor-induced activation of phosphatidylcholine-specific phospholipase C elicits mitogen-activated protein kinase phosphorylation in PC12 cells. Eur J Pharmacol, 2001. 415(2-3): p. 117-25.
56. Miranda, M., et al., Brain-Derived Neurotrophic Factor: A Key Molecule for Memory in the Healthy and the Pathological Brain. Front Cell Neurosci, 2019. 13: p. 363.
57. Herzig, S., et al., CREB controls hepatic lipid metabolism through nuclear hormone receptor PPAR-gamma. Nature, 2003. 426(6963): p. 190-3.
58. Loboda, A., et al., Role of Nrf2/HO-1 system in development, oxidative stress response and diseases: an evolutionarily conserved mechanism. Cell Mol Life Sci, 2016. 73(17): p. 3221-47.
59. Zhu, S., et al., Mechanism of NMDA Receptor Inhibition and Activation. Cell, 2016. 165(3): p. 704-14.
60. Papadia, S. and G. E. Hardingham, The dichotomy of NMDA receptor signaling. Neuroscientist, 2007. 13(6): p. 572-9.
61. Rock, D. M. and R. L. MacDonald, Spermine and related polyamines produce a voltage-dependent reduction of N-methyl-D-aspartate receptor single-channel conductance. Mol Pharmacol, 1992. 42(1): p. 157-64.
62. Satriano, J., et al., Agmatine suppresses proliferation by frameshift induction of antizyme and attenuation of cellular polyamine levels. J Biol Chem, 1998. 273(25): p. 15313-6.
63. Pegg, A. E., Spermidine/spermine-N(1)-acetyltransferase: a key metabolic regulator. Am J Physiol Endocrinol Metab, 2008. 294(6): p. E995-1010.
64. Dudkowska, M., et al., Agmatine modulates the in vivo biosynthesis and interconversion of polyamines and cell proliferation. Biochim Biophys Acta, 2003. 1619(2): p. 159-66.

1. A method for improving pain relief, antiaging effects, neuroprotective and antidepressant effects, improved vasodilatation and metabolic health, improved cellular health, and longevity, and/or reducing age-related memory loss of a subject in need thereof, the method comprising administering to the subject an effective amount of a composition comprising an isolated probiotic and arginine to increase production of agmatine in a gastrointestinal tract of the subject.

2. The method of claim 1, wherein the isolated probiotic comprises a Lactobacillus acidophilus strain.

3. The method of claim 1, wherein the isolated probiotic comprises a bacterial strain having at least 90%, preferably at least 95% sequence identity to a strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

4. The method of claim 1, wherein the composition further comprises an ingredient selected from the group consisting of a starch source, a protein source, a prebiotic source, lipid source, vitamins, sugars, salt, spices, seasonings, minerals, flavoring agents, and combinations thereof.

5. The method of claim 1, wherein the isolated probiotic comprises a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

6. The method of claim 1, wherein the isolated probiotic catalyzes the production of agmatine from arginine in the gastrointestinal tract of the subject using arginine decarboxylase (ADC) and local microbiota, wherein a concentration of the agmatine in the gastrointestinal tract of the subject is at least 20 μM at 24 hours after administering the composition.

7. (canceled)

8. The method of claim 1, wherein the isolated probiotic increases the bioavailability of Agmatine, produced from arginine in the gastrointestinal tract of the subject, through delaying the transformation of Agmatine to downstream polyamines, by at least 24 hours.

9. The method of claim 1, wherein the composition is in a form of a dried powder, and the isolated probiotic is filled into the composition.

10. The method of claim 1, wherein the isolated probiotic is active in the composition.

11-17. (canceled)

18. The method of claim 1, wherein the composition is administered to the subject in an effective amount to provide the subject a daily dose of the isolated probiotic in an amount of at least 5 million CFU and a daily dose of the arginine in an amount of 500-700 mg.

19-20. (canceled)

21. A composition comprising an isolated probiotic and arginine, the isolated probiotic comprising a bacterial strain having at least 90%, preferably at least 95% sequence identity to a strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

22. The composition of claim 21, wherein the composition further comprises an ingredient selected from the group consisting of a starch source, a protein source, a prebiotic source, lipid source, vitamins, sugars, salt, spices, seasonings, minerals, and flavoring agents.

23. The composition of claim 21, wherein the isolated probiotic comprises a live bacterial strain.

24-28. (canceled)

29. The composition of claim 21, wherein the composition is in a form of a dried powder.

30. The composition of claim 21, wherein the isolated probiotic comprises a Lactobacillus acidophilus strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

31. A method of increasing production of agmatine in a gastrointestinal tract of a subject indeed thereof, the method comprising administering to the subject a composition comprising an isolated probiotic and arginine, the isolated probiotic comprising a bacterial strain having at least 90%, preferably at least 95% sequence identity to a strain selected from the group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), and Lactobacillus acidophilus NCC 2619 (ATCC 700396).

32. The method of claim 31, wherein the isolated probiotic comprises a bacterial strain selected from a group consisting of Lactobacillus acidophilus NCC 2628 (CNCM I-2453), Lactobacillus acidophilus NCC 2766 (CNCM I-3848), Lactobacillus acidophilus NCC 2775 (CNCM I-3851), Lactobacillus acidophilus NCC 2619 (ATCC 700396), and combinations thereof.

33. The method of claim 31, wherein the composition is administered in an effective amount to produce agmatine from arginine in the gastrointestinal tract of the subject at a pH between about 4.0 and about 8.0.

34-35. (canceled)
